(19) United States
(12) Patent Application Publication
(10) Pub. No.: US 2007/0009927 A1
(43) Pub. Date: Jan. 11, 2007

(54) Methods and compositions for prenatal diagnosis of mental retardation

(76) Inventors: Ginam Cho, Narberth, PA (US); Jeffrey Golden, Wynnewood, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/392,529

(22) Filed: Mar. 29, 2006

Related U.S. Application Data
(60) Provisional application No. 60/665,978, filed on Mar. 29, 2005.

(51) Int. Cl. C12Q 1/68; C07H 21/04

(52) U.S. Cl. 435/6; 536/23.2

(57) ABSTRACT

Methods and compositions for the diagnosis of mental retardation are provided.

This application claims priority under 35 U.S.C. §119(e) to U.S. Provisional Patent Application No. 60/665,978, filed on Mar. 29, 2005. The foregoing application is incorporated by reference herein.

Pursuant to 35 U.S.C. Section 202(c), it is acknowledged that the United States Government has certain rights in the invention described herein, which was made in part with funds from the National Institutes of Health Grant No. HD26979.

### FIELD OF THE INVENTION

The present invention relates to the fields of genetic screening and molecular biology. More specifically, the invention provides compositions and methods for diagnosing mental retardation in a patient.

### BACKGROUND OF THE INVENTION

Several publications and patent documents are cited throughout the specification in order to describe the state of the art to which this invention pertains. Each of these citations is incorporated herein by reference as though set forth in full.

Development of the central nervous system requires the coordinated action of numerous signaling pathways to specify neural regions and cell types. Invertebrate and vertebrate central nervous system (CNS) development share many common principles. Both depend on signaling cascades and transcription factors to define the anterior-posterior and dorsal-ventral axes. Subsequently, these axes are further parceled into functional domains giving rise to specific neuronal subtypes. Understanding the signaling pathways required for these processes not only provides insights into normal development, but also offers insight into the pathogenesis of human disorders. The best example being holoprosencephaly, which is a defect in dorsal-ventral patterning of the forebrain (Chiang et al. (1996) Nature, 383:407-13; Golden et al. (1999) Proc. Natl. Acad. Sci., 96:2439-44).

BMP signaling plays many roles in central nervous system development including dorsal-ventral patterning and in fate specification for some neurons including forebrain cholinergic neurons (Altmann and Brivanlou (2001) Int. Rev. Cytol., 203:447-82; Mehler et al. (1997) Trends Neurosci., 20:309-17; Hebert et al. (2002) Neuron 35:1029-41; Munoz-Sanjuan and Brivanlou (2002) Nat. Rev. Neurosci., 3:271-80; Dale et al. (1997) Cell 90:257-69; Dale et al. (1999) Development 126:397-408; Lopez-Coviella et al. (2000) Science 289:313-6; Lopez-Coviella et al. (2005) Proc. Natl. Acad. Sci., 102:6984-9; Chiang et al. (1996) Nature 383:407-13; Golden et al. (1999) Proc. Natl. Acad. Sci., 96:2439-44). Signaling is initiated by dimeric ligand (BMP) binding to a type I and type II receptor complex activating the type I receptor serine/threonine kinase and resulting in phosphorylation of receptor Smads (R-Smad). Phosphorylated R-Smad interacts with Co-Smad (Smad4) and translocate to the nucleus to regulate transcription. Inhibitory Smads (I-Smad), such as Smad6 and Smad7, can block the phosphorylation of R-Smad or prevent the translocation of the R-Smad/Smad4 complex to the nucleus (Attisano and Wrana (2002) Science 296:1646-7; Feng and Derynck (2005) Annu. Rev. Cell. Dev. Biol., 21:659-693; Massague and Wotton (2000) EMBO J., 19:1745-54). Additional pathway regulation occurs in the nucleus through identical interaction of Smads with FaxH1/FAST, FoxO, Runx2, Dlx1, Hoxc-8, OAZ, GATA2, 3, 4, 5 and nuclear receptor family to enhance or repress transcription through direct DNA binding (Feng and Derynck (2005) Annu. Rev. Cell. Dev. Biol., 21:659-693). In addition, Smads are able to recruit the transcriptional coactivators or corepressors p300/CBP, P/CAF, Smad-interacting protein-1 (SIP-1), melanocyte specific gene-1 (Msg1), nuclear oncogene Ski/SnoN, smad4-interacting factor (SMIF), TGIF and Tob into the transcription machinery (Feng and Derynck (2005) Annu. Rev. Cell. Dev. Biol., 21:659-693).

### SUMMARY OF THE INVENTION

In accordance with the present invention a novel gene, Sizn1, has been identified, which when mutated, gives rise to mental retardation. More specifically, certain mutations within Sizn1, which reduce the levels of functional Sizn1 protein (e.g., by reducing the stability of the mutant protein), have been associated with mental retardation.

In one aspect of the invention, a method for diagnosing a patient as having an increased risk of having mental retardation is disclosed. An exemplary method entails providing a biological sample comprising DNA or RNA from the individual and assessing the DNA or RNA for the presence or absence of a mutation in the Sizn1 gene, wherein the presence of a Sizn1 gene mutation is correlated with the presence of mental retardation in the individual being diagnosed. Alternatively, cDNA may be generated from the mRNA of the biological sample from which the sequence of the Sizn1 gene may be analyzed.

Another exemplary diagnostic method entails providing a biological sample and assessing the level of Sizn1 protein present in the sample, wherein a decreased level of Sizn1 protein as compared to the Sizn1 protein level in a corresponding biological sample from a normal individual is correlated with the presence of mental retardation in the individual being diagnosed. In a particular embodiment, the Sizn1 protein is detected with an antibody specific for said Siz1 protein.

In yet another embodiment of the invention, the protein product encoded by the Sizn1 gene may be isolated and further assessed to determine whether a mutation in the Sizn1 gene, if present, results in an alteration in the amino acid sequence of the protein product.

Antibodies immunologically specific for Sizn1 proteins and/or mutant Sizn1 proteins are also within the scope of the present invention.

Diagnostic probes useful in the methods of the invention are also disclosed herein. Suitable probes comprise about 10-200, more preferably about 10-100 and most preferably at least 10 contiguous bases from the sequence of Sizn1 or a mutant thereof. In a particular embodiment, the probe is capable of specifically hybridizing with a mutant Sizn1 sequence, but not the wild-type Sizn1 sequence.

Also provided herein are kits for practicing the methods disclosed herein. An exemplary kit comprises the diagnostic probes described above useful for identifying alterations in the Sizn1 gene, reagents useful for nucleic acid hybridization, polymerase chain reaction (PCR), or in situ hybridization and suitable instructional materials. Another exemplary kit for detecting the presence a mutant Sizn1 protein in a biological sample is also provided. Such a kit may comprise antibodies immunologically specific for Sizn1 protein and/or mutants thereof, reagents for the detection of antibody-antigen complexes, and instructional material. Optionally, anti-Sizn1 antibodies used for this purpose may contain a detectable label or tag for used in isolating or detecting immune complexes.

### BRIEF DESCRIPTION OF THE DRAWING

FIG. 1A is a schematic of the Sizn1 protein and amino acid sequence alignment of mouse Sizn1 (SEQ ID NO: 3) and Sizn2 (SEQ ID NO: 4). The MA homologous region, Zn-finger motif, and the NLS (Nuclear Localization Sequence) are highlighted. The unique C-terminal sequence of Sizn1 used as the peptide antigen is also shown. FIG. 1B is a series of Northern blots of E12.5 dorsal (D) and ventral (V) total RNA for Sizn1, Dlx5, and GAPDH (as a control) expression. FIG. 1C is a Northern blot of Sizn1 expression in various tissues. FIGS. 1D and 1E are in situ RNA hybridizations of Sizn1 in the embryonic mouse brain. FIG. 1D is a lateral view of E12.5 telencephalon. FIG. 1E is a coronal section of E14.5 telencephalon. FIG. 1F is an alignment of Sizn1 and paraneoplastic antigen family (MA1, MA2, MA3, and MA5) and MAP-1 (MA4) (from top to bottom, SEQ ID NOs: 5 - 10).

FIGS. 2A through 2C″ are images of immunohistochemical stainings of mouse brains. FIG. 2A is an image of HEK293 cells stained with DAPI (4′,6-Diamidino-2-phenylindole), FIG. 2A′ is an image demonstrating the paraspeckle localization in the nucleus of HEK293 cells of the GFP-Sizn1 fusion protein, and FIG. 2A″ is a merge of FIGS. 2A and 2A′. FIG. 2B demonstrates DAPI staining of the P3 neonate brain, FIG. 2B′ shows the localization of Sizn1 in nuclear paraspeckles in the P3 neonate brain as detected by anti-Sizn1 antibody. FIG. 2B″ is a merge of FIGS. 2B and 2B′. The lower left boxes in FIGS. 2B, 2B′, and 2B″ are high power images corresponding to the smaller box in each image. FIG. 2C is an image of C2C12 cells stained with anti-Sizn1 antibodies, FIG. 2C′ is an image of C2C12 cells stained with anti-PML antibodies. FIG. 2C″ is a merge of the images in FIGS. 2C and 2C′.

FIG. 3A contains images of a Western blot (left panel) and an autoradiograph (right panel) of a GST-pull down assay showing that GST-Sizn1 protein purified from E. coli binds with in vitro translated Smad1 protein. FIG. 3B contains images of Western blots with anti-FLAG antibody (top panel) and anti-myc antibody (middle panel) of immunoprecipitations of HEK293T cells. FIG. 3B also contains an image of a Western blot with anti-FLAG antibody of crude extracts of HEK293T cells (bottom panel). FIG. 3C is a graph of the luciferase activity of C2C12 cells cotransfected with SBEx4-luciferase reporter, a constitutively active form of the BMPR1a, and varying concentrations of Sizn1 as indicated along the bottom of the graph (n=10). FIG. 3D is a graph of the luciferase activity of the Gal4×5-luciferase reporter assay showing Sizn1 enhances Smad1 function (n=8). FIG. 3E is a graph of a reporter assay with SBEx4-luciferase gene which is co-activated in the presence of BMP-2 (n =6). FIG. 3F contains images of Western blots of immunoprecipitations (left panels) with anti-CBP antibody or a control antibody (rabbit IgG) and crude extracts of C2C12 cells transfected with Sizn1 expression constructs (right panels). The Western blots were performed with either anti-CBP antibody (top panels) or anti-myc antibodies (bottom panels). FIG. 3G is a graph of the luciferase activity of C2C12 cells transfected with certain combinations of the SBEx4-luciferase reporter gene, a constitutively active form of the BMPR1a, CBP and Sizn1 as indicated along the bottom of the graph (n=8). FIG. 3H contains images of Western blots with anti-FLAG antibody (top panel) and anti-myc antibody (middle panel) of immunoprecipitations with anti-Sizn1 of HEK293T cells. FIG. 3H also contains an image of a Western blot with anti-FLAG antibody of crude extracts of HEK293T cells (bottom panel). The numbers in the bottom line of FIG. 3H refer to specific Smad proteins (e.g., 2 is Smad2).

FIG. 4A is an immunostained image depicting Sizn1 and AChE (acetyl cholinesterase) localization in a coronal section of an E13.5 mouse brain. FIG. 4B provides graphs of the real time RT-PCR of total RNAs of Sizn1 or ChAT prepared 5 days after plating cells in the presence or absence of BMP-2 (10 ng/ml). Scores (average±s.e.m.) were calculated by normalization to first sample. bFGF (20 ng/ml) was added to each sample to maintain high numbers of precursor cell. FIG. 4C is a graph of ChAT-luciferase reporter gene activity in C2C12 cells activated by BMP-2 in the presence of Sizn1 (n=4). FIG. 4D provides images of Western blots (left) of Sizn1 in SN56 cell after transfection of ShRNA expression constructs (Lane 1 is negative control; Lane 2-4 are 3 distinct shRNA expression constructs designed to unique sites within Sizn1 coding region). Target sequence II (lane 3) was used for subsequent experiments. The right panels of FIG. 4D are images of Northern blot analyses of VaChT in primary septal cell culture.

FIGS. 5A contains a chart of the known pedigree of family K8923 with XLMR (c.1031C>T). Automated sequence chromatograms of SIZN1 exon 4 from a control male (SEQ ID NO: 11) and from four affected males (SEQ ID NO: 12) and a carrier female (SEQ ID NOs: 11 and 12) in family K8923 showing a “C” to “T” alteration at nucleotide 1031 (c.1031C>T). CMS2604 is an obligate carrier and is heterozygous for the alteration. Mutant alleles are boxed. This alteration is predicted to cause a p.T344I missense mutation in SIZN1. FIG. 5B provides a chart of the pedigree of family K9264 with XLMR (c.19C>T). Mutation analysis in family K9264 and two unrelated patients with MR (CMS4957 and CMS7492) was performed. A 622-bp SIZN1 exon 4 PCR product amplified from genomic DNA was digested with Nsp1 restriction enzyme (generated by the c.19C>T nucleotide alteration) to distinguish between the normal (622-bp) and the mutant (555-bp) alleles. A 67-bp fragment generated by the restriction digestion in affected individuals is not shown. Restriction digestion analysis shows affected males carry the c.19C>T mutation. This alteration is predicted to cause a p.R7C missense mutation in SIZN1. Squares, males; circles, females; filled square, affected male; circle with dot, unaffected female carrying one copy of a normal and a mutant allele. FIG. 5C contains a graph of the luciferase activity of the SBEx4 reporter assay and the indicated constructs and a Western blot analysis with anti-Sizn1 antibody. FIG. 5D contains a graph of a pulse-chase analysis (n=3) of Sizn1 proteins and images of autoradiographs of wild-type Sizn1 (top panel) and mutant Sizn1 (bottom panel) immunoprecipitated with anti-myc antibody from each time points.

FIG. 6A provides the nucleotide sequence of human Sizn1 (SEQ ID NO: 1). FIG. 6B provides the amino acid sequence of human Sizn1 (SEQ ID NO: 2).

FIG. 7A provides images of a Western blot analysis of representative of anti-Ma2 paraneoplastic encephalitis and normal controls. Three of 5 anti-Ma2 patients showed SIZN1 positive reaction against Sizn1-His protein as representative. Lane (+): rabbit serum against Sizn1 (diluted 1:1,500). Lanes 1-5: patients with anti-Ma2 encephalitis (3 SIZN1 positive, 2 SIZN1 negative). Lanes 6-7: negative controls. The detected bands were 55 kD, which is bigger than wild type because of the His-tag fusion at N and C-terminal of Sizn1.

FIG. 7B provides saggital (left panel) and coronal (right panel) T2 weighted MRI images from patient #2. Increase intensity (white areas) show abnormal signal in the septal region (S), dicephalon (D), upper mesencephalon (M), and amygdale bilaterally (A).

FIG. 8A provides a diagram of the Sizn1 deletion mutants and a summary of the functional activities of the deletion mutants. FIG. 8B provides images of various immunostains. The left side indicates the mutant names and the top side indicates the staining methods. FIG. 8C provides images of the subcellular localization of Sizn1 deletion mutants. FIG. 8D provides the results of the reporter gene assays with the deletion mutants (n=5). FIGS. 8E and 8F provide images of immunoblots showing the results of the GST-pulldown assay with CBP deletion mutants (8E) and Sizn1 deletion mutants (8F) indicating that the N-terminal region of Sizn1 (1-250) can bind to the N-terminal region of CBP (1-770). FIG. 8G provides images of immunoblots of GST-pulldown assays of the binding of all of the Sizn1 deletion mutants to Smad1.

### DETAILED DESCRIPTION OF THE INVENTION

The present invention relates generally to the use of mutations in the Sizn1 as an indicator of mental retardation. In a particular embodiment, the identification of mutations in Sizn1 can be used for prenatal diagnosis of individuals for mental retardation. The invention also relates to therapies for the treatment of mental retardation due to the presence of at least one mutation in the Sizn1 gene. Such treatment modalities include gene therapy, protein replacement therapy and administration protein mimetics. The invention further relates to the screening of drugs which may have therapeutic value. Finally, the invention relates to the screening of the Sizn1 gene for mutations, which are useful for diagnosing a predisposition to mental retardation.

The present invention provides an isolated polynucleotide comprising a Sizn1 encoding nucleic acid molecule. Recombinant constructs comprising such an isolated polynucleotide (e.g., a recombinant construct suitable for expression in a transformed host cell) are also provided.

Also provided by the present invention are methods for detecting a polynucleotide comprising the Sizn1 nucleic acid molecule or a mutant thereof. Such methods may comprise the step of amplifying the Sizn1 encoding nucleic acid molecule from a biological sample by PCR amplification. The method is useful for diagnosis or prognosis of mental retardation in an individual.

The present invention also provides isolated antibodies, preferably monoclonal antibodies, which specifically bind to a Sizn1 polypeptides.

The present invention also provides kits for detecting a Sizn1 polynucleotide, the kits may comprise Sizn1 probes packaged in a suitable container and instructions for its use.

The present invention further provides methods of screening the Sizn1 gene to identify mutations associated with mental retardation. Such methods may further comprise the step of amplifying the Sizn1 gene by PCR amplification, sequencing the amplified nucleic acid molecule, and comparing the sequence of the amplified nucleic acid molecule with wild-type Sizn1.

The present invention further provides methods of screening drugs for therapy and to identify suitable drugs for restoring Sizn1 gene product function.

Finally, the present invention provides the means necessary for production of gene-based therapies directed at aberrant cells associated with mental retardation. These therapeutic agents may take the form of polynucleotides comprising the Sizn1 gene placed in appropriate vectors or delivered to target cells in more direct ways such that the function of the Sizn1 protein is reconstituted. Therapeutic agents may also take the form of Sizn1 proteins.

I. Definitions

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

A “replicon” is any genetic element, for example, a plasmid, cosmid, bacmid, plastid, phage or virus, which is capable of replication largely under its own control. A replicon may be either RNA or DNA and may be single or double stranded. Generally, a “viral replicon” is a replicon which contains the complete genome of the virus. A “sub-genomic replicon” refers to a viral replicon that contains something less than the full viral genome, but is still capable of replicating itself. For example, a sub-genomic replicon may contain most of the genes encoding for the non-structural proteins of the virus, but not most of the genes encoding for the structural proteins.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The terms “percent similarity”, “percent identity” and “percent homology” when referring to a particular sequence are used as set forth in the University of Wisconsin GCG software program.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

Polymerase chain reaction (PCR) has been described in U.S. Pat. Nos. 4,683,195, 4,800,195, and 4,965,188, the entire disclosures of which are incorporated by reference herein.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., 1989, Molecular Cloning: A Laboratory Manual, Cold Spring Harbor Laboratory Press):
Tₘ=81.5° C. +16.6 Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tm of a DNA duplex decreases by 1−1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5× Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5× Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, or the addition of stabilizers.

The term “gene” refers to a nucleic acid comprising an open reading frame encoding a polypeptide, including both exon and (optionally) intron sequences. The nucleic acid may also optionally include non-coding sequences such as promoter or enhancer sequences. The term “intron” refers to a DNA sequence present in a given gene that is not translated into protein and is generally found between exons.

As used herein, the term “solid support” refers to any solid or stationary material to which reagents such as antibodies, antigens, and other test components can be attached. Examples of solid supports include, without limitation, microtiter plates (or dish), microscope (e.g. glass) slides, coverslips, beads, cell culture flasks, chips (for example, silica-based, glass, or gold chip), membranes, particles (typically solid; for example, agarose, sepharose, polystyrene or magnetic beads), columns (or column materials), and test tubes. Typically, the solid supports are water insoluble.

As used herein, an “instructional material” includes a publication, a recording, a diagram, or any other medium of expression which can be used to communicate the usefulness of the composition of the invention for performing a method of the invention.

The term “mental retardation,” as used herein, is broadly defined as a significantly sub-average general intellectual functioning that is accompanied by significant limitations in adaptive functioning. Mental retardation can be categorized as mild mental retardation (IQ from about 50-70) or as severe mental retardation (IQ less than 50).

As used herein, the term “biological sample” refers to a subset of the tissues of a biological organism, its cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen).

A “mutation” is any alteration in the Sizn1 gene which alters function or expression of Sizn1 gene products, such as mRNA and the encoded for protein.

II. Nucleic Acid Molecules

Nucleic acid molecules encoding the Sizn1 proteins of the invention may be prepared by three general methods: (1) synthesis from appropriate nucleotide triphosphates, (2) isolation from biological sources, and (3) mutation of nucleic acid molecule encoding Sizn1 protein. These methods utilize protocols well known in the art. The availability of nucleotide sequence information, such as the sequences provided herein, enables preparation of an isolated nucleic acid molecule of the invention by oligonucleotide synthesis. Synthetic oligonucleotides may be prepared by the phosphoramidite method employed in the Applied Biosystems 38A DNA Synthesizer or similar devices. The resultant construct may be purified according to methods known in the art, such as high performance liquid chromatography (HPLC). Long, double-stranded polynucleotides may be synthesized in stages, due to any size limitations inherent in the oligonucleotide synthetic methods.

Nucleic acid sequences encoding the Sizn1 proteins of the invention may be isolated from appropriate biological sources using methods known in the art. In one embodiment, a cDNA clone is isolated from a cDNA expression library of human origin. In an alternative embodiment, utilizing the sequence information provided by the cDNA sequence, human genomic clones encoding altered Sizn1 proteins may be isolated. Additionally, cDNA or genomic clones having homology with human and mouse Sizn1 may be isolated from other species using oligonucleotide probes corresponding to predetermined sequences within the human and mouse Sizn1 encoding nucleic acids.

An exemplary nucleotide sequence encoding Sizn1 is SEQ ID NO: 1. A Sizn1 nucleotide sequence may have 75%, 80%, 85%, 90%, 95%, 97%, or 99% homology with SEQ ID NO: 2.

In accordance with the present invention, nucleic acids having the appropriate level of sequence homology with a nucleic acid molecule encoding Sizn1 may be identified by using hybridization and washing conditions of appropriate stringency.

Nucleic acids of the present invention may be maintained as DNA in any convenient vector. Sizn1 encoding nucleic acid molecules of the invention include cDNA, genomic DNA, RNA, and fragments thereof which may be single- or double-stranded. Thus, this invention provides oligonucleotides having sequences capable of hybridizing with at least one sequence of a nucleic acid molecule of the present invention.

Also contemplated in the scope of the present invention are oligonucleotide probes which specifically hybridize with the mutated Sizn1 nucleic acid molecules of the invention while not hybridizing with the wild type sequence under high or very high stringency conditions. Primers capable of specifically amplifying Sizn1 encoding nucleic acids described herein are also contemplated herein. As mentioned previously, such oligonucleotides are useful as probes and primers for detecting, isolating or amplifying altered Sizn1 genes.

It will be appreciated by persons skilled in the art that variants (e.g., allelic variants) of Sizn1 sequences exist in the human population, and must be taken into account when designing and/or utilizing oligonucleotides of the invention. Accordingly, it is within the scope of the present invention to encompass such variants, with respect to the Sizn1 sequences disclosed herein or the oligonucleotides targeted to specific locations on the respective genes or RNA transcripts. Accordingly, the term “natural allelic variants” is used herein to refer to various specific nucleotide sequences of the invention and variants thereof that would occur in a human population. The usage of different wobble codons and genetic polymorphisms which give rise to conservative or neutral amino acid substitutions in the encoded protein are examples of such variants. Such variants would not demonstrate altered Sizn1 activity or protein levels. Additionally, the term “substantially complementary” refers to oligonucleotide sequences that may not be perfectly matched to a target sequence, but such mismatches do not materially affect the ability of the oligonucleotide to hybridize with its target sequence under the conditions described.

III. Proteins

Sizn1 proteins of the present invention may be prepared in a variety of ways, according to known methods. The proteins may be purified from appropriate sources, e.g., transformed bacterial or animal cultured cells or tissues, by immunoaffinity purification. The availability of nucleic acid molecules encoding Sizn1 protein enables production of the protein using in vitro expression methods and cell-free expression systems known in the art. In vitro transcription and translation systems are commercially available, e.g., from Promega Biotech (Madison, Wis.) or Gibco-BRL (Gaithersburg, Md.).

Alternatively, larger quantities of Sizn1 protein may be produced by expression in a suitable prokaryotic or eukaryotic system. For example, part or all of a DNA molecule encoding for Sizn1 may be inserted into a plasmid vector adapted for expression in a bacterial cell, such as E. coli. Such vectors comprise the regulatory elements necessary for expression of the DNA in the host cell positioned in such a manner as to permit expression of the DNA in the host cell. Such regulatory elements required for expression include promoter sequences, transcription initiation sequences and, optionally, enhancer sequences.

Sizn1 protein produced by gene expression in a recombinant procaryotic or eukaryotic system may be purified according to methods known in the art. A commercially available expression/secretion system can be used, whereby the recombinant protein is expressed and thereafter secreted from the host cell, and readily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope or the hemagglutinin epitope. Such methods are commonly used by skilled practitioners.

Sizn1 protein of the invention, prepared by the aforementioned methods, may be analyzed according to standard procedures. For example, such protein may be subjected to amino acid sequence analysis, according to known methods.

An exemplary amino acid sequence of Sizn1 is SEQ ID NO: 2. A Sizn1 amino acid sequence may have 75%, 80%, 85%, 90%, 95%, 97%, or 99% homology with SEQ ID NO: 2.

The present invention also provides antibodies capable of immunospecifically binding to proteins of the invention. Polyclonal antibodies directed toward Sizn1 protein and mutants thereof may be prepared according to standard methods. In a preferred embodiment, monoclonal antibodies are prepared, which react immunospecifically with the various epitopes of the Sizn1 protein. Monoclonal antibodies may be prepared according to general methods known in the art. Polyclonal or monoclonal antibodies that immunospecifically interact with wild-type and/or mutant Sizn1 proteins can be utilized for identifying and purifying such proteins. For example, antibodies may be utilized for affinity separation of proteins with which they immunospecifically interact. Antibodies may also be used to immunoprecipitate proteins from a sample containing a mixture of proteins and other biological molecules.

IV. Screening Methods

There are numerous methods for detecting a mutation in a gene (see, in general, Ausubel et al. (1998) Current Protocols in Molecular Biology, John Wiley & Sons, New York. Exemplary approaches for detecting alterations in Sizn1 encoding nucleic acids include, without limitation:

a) comparing the sequence of nucleic acid molecules in a sample from a patient with the wild-type Sizn1 nucleic acid sequence to determine whether the sample from the patient contains mutations;

b) determining the presence, in a sample from a patient, of the polypeptide encoded by the Sizn1 gene and, if present, determining whether the polypeptide is mutated and/or is expressed at the normal level;

c) using DNA restriction mapping to compare the restriction pattern produced when a restriction enzyme cuts a sample of nucleic acid from the patient with the restriction pattern obtained from normal Sizn1 gene or from known mutations thereof;

d) using a specific binding member capable of binding to a Sizn1 nucleic acid sequence (either normal sequence or known mutated sequence), the specific binding member comprising either nucleic acid molecules hybridizable with the Sizn1 sequence or substances comprising an antibody domain with specificity for Sizn1 nucleic acid sequence (either normal sequence or known mutated sequence) or the polypeptide encoded by it, the specific binding member being labeled so that binding of the specific binding member to its binding partner is detectable; and

e) using PCR involving one or more primers based on normal or mutated Sizn1 gene sequence to screen for normal or mutant Sizn1 gene in a sample from a patient.

More specific examples of screening methods are provided hereinbelow.

In certain embodiments for screening for mutant Sizn1 encoding nucleic acid molecules, the Sizn1 nucleic acid in the sample will initially be amplified, e.g. using PCR, to increase the amount of Sizn1 nucleic acid molecules as compared to other sequences present in the sample. This allows the target Sizn1 sequences to be detected with a high degree of sensitivity if they are present in the sample. This initial step may be avoided by using highly sensitive array techniques.

Hitherto uncharacterized variations in the Sizn1 gene can be identified and localized to specific nucleotides by comparison of nucleic acids from an individual with mental retardation with an unaffected individual. Comparison with a relative may be preferred because the possibility of other polymorphic differences between the patient and person being compared, not related to the mental retardation phenotype, is lower. Various screening methods are suitable for this comparison including, but not limited to, direct DNA sequencing, single strand conformation polymorphism analysis (SSCP), conformation shift gel electrophoresis (CSGE), heteroduplex analysis (HA), chemical cleavage of mismatched sequences (CCMS), denaturing gradient gel electrophoresis (DGGE), temperature gradient gel electrophoresis (TGGE), denaturing high performance liquid chromatography (dHPLC), ribonuclease cleavage, carbodiimide modification, and microarray analysis. See, e.g., Cotton (1993) Mutation Res. 285:125-144. Comparison can be initiated at either cDNA or genomic level. Initial comparison is often easier at the cDNA level because of its shorter size. Corresponding genomic changes are then identified by amplifying and sequencing a segment from the genomic exon including the site of change in the cDNA. In some instances, there is a simple relationship between genomic and cDNA changes. That is, a single base change in a coding region of genomic DNA gives rise to a corresponding changed codon in the cDNA. In other instances, the relationship between genomic and cDNA changes is more complex. Thus, for example, a single base change in genomic DNA creating an aberrant splice site can give rise to deletion of a substantial segment of cDNA.

The preceding methods serve to identify particular genetic changes responsible for mental retardation. Once a change has been identified, individuals can be tested for that change by various methods. These methods include direct sequencing, allele-specific oligonucleotide hybridization, allele-specific amplification, ligation, primer extension and artificial introduction of extension sites (see Cotton, supra). Of course, the methods noted above, for analyzing uncharacterized variations can also be used for detecting characterized variations. Certain methods are described in more detail below.

Mutational Analysis/Conformation Sensitive Gel Electrophoresis (CSGE).

Conformation sensitive gel electrophoresis (CSGE) can be performed using standard protocols (Ganguly, A. et al. (1993) PNAS 90:10325-10329). PCR products corresponding to all altered migration patterns (shifts) can be purified and sequenced.

Isolation and Amplification of DNA

Samples of patient genomic DNA can be isolated from any suitable cell, fluid, or tissue sample. The cells can be obtained from solid tissue as from a fresh or preserved organ or from a tissue sample or biopsy. The sample can contain compounds which are not naturally intermixed with the biological material such as preservatives, anticoagulants, buffers, fixatives, nutrients, antibiotics, or the like.

Methods for isolation of genomic DNA from these various sources are described in, for example, Kirby, DNA Fingerprinting, An Introduction, W. H. Freeman & Co. New York (1992). Genomic DNA can also be isolated from cultured primary or secondary cell cultures or from transformed cell lines derived from any of the aforementioned tissue samples.

Samples of patient RNA can also be used. RNA can be isolated from tissues expressing the Sizn1 gene as described in Sambrook et al., supra. RNA can be total cellular RNA, mRNA, poly A+ RNA, or any combination thereof. RNA can be reverse transcribed to form DNA which is then used as the amplification template, such that the PCR indirectly amplifies a specific population of RNA transcripts. See, e.g., Sambrook, supra, Kawasaki et al., Chapter 8 in PCR Technology, (1992) supra, and Berg et al. (1990) Hum. Genet. 85:655-658.

PCR Amplification

The most common means for amplification is polymerase chain reaction (PCR), as described in U.S. Pat. Nos. 4,683,195, 4,683,202, and 4,965,188. To amplify a target nucleic acid sequence in a sample by PCR, the sequence must be accessible to the components of the amplification system. Methods of isolating target DNA by crude or fine extraction are known in the art. See, e.g., Higuchi, “Simple and Rapid Preparation of Samples for PCR”, in PCR Technology, Ehrlich, H. A. (ed.), Stockton Press, New York, and Miller et al. (1988) Nucleic Acids Res. 16:1215. Notably, kits for the extraction of DNA for PCR are also readily available.

Allele Specific PCR

Allele-specific PCR differentiates between target regions differing in the presence or absence of a mutation. PCR amplification primers are chosen which bind only to certain alleles of the target sequence, e.g., a Sizn1 gene comprising a mutation. Thus, for example, amplification products are generated from those samples which contain the primer binding sequence and no amplification products are generated in samples without the primer binding sequence. This method is described by Gibbs (1989) Nucleic Acid Res. 17:12427-2448.

Allele Specific Oligonucleotide Screening Methods

Further diagnostic screening methods employ the allele-specific oligonucleotide (ASO) screening methods, as described by Saiki et al. (1986) Nature 324:163-166. Oligonucleotides with one or more base pair mismatches are generated for any particular Sizn1. ASO screening methods detect mismatches between variant target genomic or PCR amplified DNA and non-mutant oligonucleotides, showing decreased binding of the oligonucleotide relative to a mutant oligonucleotide. Oligonucleotide probes can be designed that under low stringency will bind to both wild-type and mutant forms of Sizn1, but which at higher stringency, bind to the form to which they correspond. Alternatively, stringency conditions can be devised in which an essentially binary response is obtained, i.e., an ASO corresponding to a mutant form of the Sizn1 gene will hybridize to that allele and not to wild-type Sizn1.

Ligase Mediated Allele Detection Method

Target regions of a patient can be compared with target regions in unaffected individuals by ligase-mediated allele detection. See, e.g., Landegren et al. (1988) Science 241:1077-1080. Ligase may also be used to detect point mutations in the ligation amplification reaction described in Wu et al. (1989) Genomics 4:560-569. The ligation amplification reaction (LAR) utilizes amplification of specific DNA sequence using sequential rounds of template dependent ligation as described in Wu et al. and Barany (1990) PNAS 88:189-193.

Denaturing Gradient Gel Electrophoresis

Amplification products generated using the polymerase chain reaction can be analyzed by the use of denaturing gradient gel electrophoresis. Different mutations/alleles can be identified based on the different sequence-dependent melting properties and electrophoretic migration of DNA in solution. Differentiation between mutant and wild-type sequences based on specific melting domain differences can be assessed using polyacrylamide gel electrophoresis, as described, for example, in Chapter 7 of Erlich, ed., PCR Technology, Principles and Applications for DNA Amplification, W. H. Freeman and Co, New York (1992).

Generally, a target region to be analyzed by denaturing gradient gel electrophoresis is amplified using PCR primers flanking the target region. The amplified PCR product is applied to a polyacrylamide gel with a linear denaturing gradient as described, for example, in Myers et al. (1986) Meth. Enzymol. 155:501-527 and Myers et al., in Genomic Analysis, A Practical Approach, K. Davies Ed. IRL Press Limited, Oxford, pp. 95-139 (1988). The electrophoresis system is maintained at a temperature slightly below the Tm of the melting domains of the target sequences.

In an alternative method of denaturing gradient gel electrophoresis, the target sequences may be initially attached to a stretch of GC nucleotides, termed a GC clamp, as described, for example, in Chapter 7 of Erlich, supra. Preferably, at least 80% of the nucleotides in the GC clamp are either guanine or cytosine. Preferably, the GC clamp is at least 30 bases long. This method is particularly suited to target sequences with high melting temperatures.

Temperature Gradient Gel Electrophoresis

Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis.

Single-Strand Conformation Polymorphism Analysis

Target sequences or mutants at the Sizn1 locus can be differentiated using single-strand conformation polymorphism analysis, which identifies base differences by alteration in electrophoretic migration of single stranded PCR products, as described, for example, in Orita et al. (1989) PNAS 86:2766-2770 and Example I hereinbelow. Amplified PCR products can be generated as described above, and heated or otherwise denatured, to form single stranded amplification products. Single-stranded nucleic acids may refold or form secondary structures which are partially dependent on the base sequence. Thus, electrophoretic mobility of single-stranded amplification products can detect base-sequence difference between alleles or target sequences. Chemical or Enzymatic Cleavage of Mismatches Differences between target sequences can also be detected by differential chemical cleavage of mismatched base pairs, as described, for example, in Grompe et al. (1991) Am. J. Hum. Genet. 48:212-222. In another method, differences between target sequences can be detected by enzymatic cleavage of mismatched base pairs, as described, for example, in Nelson et al. (1993) Nature Genetics 4:11-18. Briefly, genetic material from a patient and an unaffected individual may be used to generate mismatch free heterohybrid DNA duplexes. As used herein, “heterohybrid” means a DNA duplex strand comprising one strand of DNA from one person, usually the patient, and a second DNA strand from another person, usually an unaffected individual. Positive selection for heterohybrids free of mismatches allows determination of small insertions, deletions or other polymorphisms that may be associated with mental retardation.

Non-PCR Based DNA Diagnostics

The identification of a DNA sequence linked to Sizn1 can made without an amplification step, based on polymorphisms including restriction fragment length polymorphisms in a patient and a normal individual. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes are preferably labeled directly or indirectly, such that by assaying for the presence or absence of the probe, one can detect the presence or absence of the target sequence. Direct labeling methods include radioisotope labeling, such as with ³²P or ³⁵S. Indirect labeling methods include fluorescent tags, biotin complexes which may be bound to avidin or streptavidin, or peptide or protein tags. Visual detection methods include, without limitation, photoluminescents, chemoluminescence, horse radish peroxidase, alkaline phosphatase, and the like.

According to another aspect of the invention, methods of screening drugs to identify suitable drugs for restoring Sizn1 function are provided.

One technique for drug screening involves the use of host eukaryotic cell lines or cells which have a mutant Sizn1 gene. These host cell lines or cells are defective at the Sizn1 polypeptide level. The host cell lines or cells are placed in the presence of a test compound. The restoration of BMP signaling or increased Sizn1 protein levels (see Example 1), for example, in the presence of the test compound suggests the compound is capable of restoring Sizn1 function to the cells.

V. Therapeutics

The discovery that mutations in the Sizn1 gene give rise to mental retardation facilitates the development of pharmaceutical compositions useful for treatment and diagnosis of these syndromes and conditions. These compositions may comprise therapeutic agent (such as an agent identified by the above screens or a nucleic acid molecule encoding for wild-type Sizn1) in a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, and intraperitoneal routes.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

The methods may also be used to advantage for in utero screening of fetuses for the presence of a mutant Sizn1. Identification of such variations offers the possibility of gene therapy. For couples known to be at risk of giving rise to affected progeny, diagnosis can be combined with in vitro reproduction procedures to identify an embryo having wild type Sizn1 before implantation. Screening children shortly after birth is also of value in identifying those having a mutant Sizn1 gene. Early detection allows administration of appropriate treatment.

As a further alternative, the nucleic acid encoding the wild-type Sizn1 polypeptide could be used in a method of gene therapy, to treat a patient who is unable to synthesize the active protein to normal levels, thereby restoring normal Sizn1 function.

Vectors, such as viral vectors have been used in the prior art to introduce genes into a wide variety of different target cells. Typically the vectors are exposed to the target cells so that transformation can take place in a sufficient proportion of the cells to provide a useful therapeutic or prophylactic effect from the expression of the desired polypeptide. The transfected nucleic acid may be permanently incorporated into the genome of each of the targeted cells, providing long lasting effect, or alternatively the treatment may have to be repeated periodically. A variety of vectors for gene therapy, both viral vectors and plasmid vectors, are known in the art.

VI. Kits

The present invention also includes kits for the practice of the methods of the invention. The kits may comprise at least one oligonucleotide which specifically hybridizes with mutant Sizn1 encoding nucleic acid molecules, reaction buffers, and instructional material. Optionally, the at least one oligonucleotide contains a detectable tag. Certain kits may contain two such oligonucleotides, which serve as primers to amplify at least part of the Sizn1 gene. The part selected for amplification can be a region from the Sizn1 gene that includes a site at which a mutation is known to occur. Some kits contain a pair of oligonucleotides for detecting precharacterized mutations. Alternatively, the kit may comprise primers for amplifying at least part of the Sizn1 gene to allow for sequencing and identification of mutant Sizn1 nucleic acid molecules. The kits of the invention may also contain components of the amplification system, including PCR reaction materials such as buffers and a thermostable polymerase. In other embodiments, the kit of the present invention can be used in conjunction with commercially available amplification kits, such as may be obtained from GIBCO BRL (Gaithersburg, Md.) Stratagene (La Jolla, Calif.), Invitrogen (San Diego, Calif.), Schleicher & Schuell (Keene, N.H.), Boehringer Mannheim (Indianapolis, Ind.). The kits may optionally include instructional material, positive or negative control reactions, templates, or markers, molecular weight size markers for gel electrophoresis, and the like.

Kits of the instant invention may also comprise antibodies immunologically specific for Sizn1 protein and/or mutants thereof and instructional material. Optionally the antibody contains a detectable tag. The kits may optionally include buffers for forming the immunocomplexes, agents for detecting the immunocomplexes, instructional material, solid supports, positive or negative control samples, molecular weight size markers for gel electrophoresis, and the like. VII. Paraneoplastic neurological disease (PND) In accordance with the instant invention, antibodies to Sizn1 were uniquely detected in patients with a PND that is characteristically associated with severe memory deficits and behavioral and cognitive abnormalities (Dalmau et al. (2004) Brain 127:1831-1844). These results reflect an immune mediated dysfunction of septal cholinergic neurons. Although the exact mechanism by which antibodies to nuclear proteins result in autoimmune disorders such as PND, it is clear that antibodies to a variety of nuclear proteins participate in the pathogenesis of many autoimmune disorders such as PND (anti-Hu;, anti-Yo;, anti-Ri; and anti-Ma; see Dalmau and Posner (1999) Arch. Neurol., 56:405-408). Other dominant symptoms and MRI findings of this PND result from dysfunction referable to the limbic, diencephalic and upper mesencephalic brain regions, all of which overlap with Sizn1 expression.

In one embodiment of the instant invention, methods are provided for the diagnosis of MA-related PNDs. The methods comprise the detection of Sizn1 antibodies in a patient, wherein the detection of Sizn1 antibodies indicates the presence of a PND, particularly an MA-related PND.

In another embodiment, methods are provided for screening and identifying agents for their ability to treat a PND, particularly an MA-related PND. These methods comprise contacting Sizn1 antibodies with one or more agents to identify those agents which neutralize the Sizn1 antibody, i.e., prevent the Sizn1 antibody from binding Sizn1. In a particular embodiment, the Sizn1 antibody is isolated from a patient suffering from a PND. In another embodiment, the agents screened comprise small molecule inhibitors, anti-idiotypic antibodies, or peptides of Sizn1, particularly short peptide of between about 10 and about 50 amino acids. In yet another embodiment, the ability of the agent to block the binding of the Sizn1 antibody to Sizn1 is determined with full-length Sizn1 protein or fragments thereof. Such Sizn1 fragments are preferably between about 5 and about 400 amino acids, between about 10 and about 200 amino acids, between about 10 and about 100 amino acids, or between about 10 and about 25 amino acids. The Sizn1 fragments preferably comprise at least one immunologically recognized epitope. Agents identified by the above screening method may be subsequently administered to patient suffering from a PND, particularly an MA-related PND, for treatment of the PND.

The following examples are provided to illustrate various embodiments of the present invention. They are not intended to limit the invention in any way.

### EXAMPLE 1

Methods

The following methods were employed to perform the experiments described hereinbelow.

Subtractive Hybridization Screening

mRNA was isolated from dorsal and ventral telencephalon of E11.5 and E12.5 mouse embryos, using the oligo dT cellulose power (FastTrack 2.0; Invitrogen, Carlsbad, Calif.). For subtractive hybridization, the PCR-select cDNA subtraction kit was used according to the manufacturer's instructions (Clontech, Palo Alto, Calif.). Subtracted clones were amplified and PCR products were subcloned into pBluescript SKII (Strategene, La Jolla, Calif.). For differential screening, each clone was spotted onto two separate nitrocellulose membranes. The membranes were separately hybridized with random primed cDNA probes from dorsal or ventral cDNA. Clones showing differential expression were sequenced. The full length clone of Sizn was obtained by screening a mouse E12.5 cDNA library (provide by Doug Epstein), using the partial cDNA of Sizn1 derived from the subtractive hybridization screen.

Cell Culture, Transfection and Luciferase Assay

HEK293T and C2C12 cell lines were cultured in DMEM containing 10% fetal bovine serum (FBS; HyClone, Logan, UT; 15% FBS for C2C12 cells). The cells were maintained in humidified atmosphere with 5% CO₂ at 37° C. C2C12 cells (5×10⁴ cells/well in six-well tissue culture plates) were transfected with various combinations of the following plasmids using FuGene6 (Roche, Indianapolis, Ind.); reporter constructs (SBE×4luc; 0.1 μg), β-galactosidase expression vector driven by the CMV promoter (CMV-β-gal; 0.05 μg), constitutive active BMPR1a (QD; 0.5 μg) or CBP expression construct (500 ng). Empty vectors for each construct were used as control. In the Gal4×5 luciferase assays, the reporter construct (Gal4×5luc; 100 ng), β-galactosidase expression vector driven by CMV promoter (CMV-β-gal; 0.05 μg), Gal4DB-Smad1 expression construct and pMIW/Sizn1-myc (each 500 ng) were used. Twenty-four hours after transfection, new media was added and culture continued for another 24 hours. Cell extracts were prepared by Promega Lysis Buffer followed by centrifugation. Luciferase activity was measured by Promega luciferase assay system (Madison, Wis.). β-galactosidase activity was measured to standardize transfection efficiency. The luciferase reporter assay was performed in duplicate and then plotted after calculating mean and standard deviation.

DNA Construction

pCMV/Sizn1-GFP was generated by subcloning the mouse Sizn1 coding region into pcDNA3-CTGFP (Invitrogen). pMIWIII/Sizn1-myc was generated by subcloning the PCR product containing Sizn1 coding region into at EcoRV of pMIWIII (Lim et al. (2005) Mech. Dev., 122:603-620). pGSTSizn1 was constructed by subcloning Sizn1 PCR product into pGEX-5T (Amersham, Piscataway, N.J.). Flag-Smad1 expression constructs were obtained from Dr. Miyazono (JFCF cancer institute, Japan). CBP expression constructs was obtained from Dr. Blobel (The Children's Hospital of Philadelphia).

Yeast Two Hybrid Screening

The full-length Sizn1 coding sequence was PCR amplified and cloned into the EcoRV site of pGBKT7 (Clontech) to generate the Sizn bait construct. The Yeast cell line, AH109(MATa) containing the Sizn bait construct was mated with Y187(MAT a) cells pre-transformed with mouse E11 embryo two hybrid system library Matchmaker3 (Clontech) to screen for interaction partners of Sizn1 according to the manufacturer's instruction. Plasmids from positive yeast clones were purified, transformed to E.coli and sequenced.

GST Pull-Down Assay

GST-fusion proteins were expressed in the E. coli and bound to glutathionesepharose 4B beads (Pharmachia Biotech, Piscataway, N.J.). The in vitro translated proteins were then incubated with beads in TNE buffer (20 mM Tris-HCl, pH7.4, 150 mM NaCl, 1 mM EDTA, and 0.5% NP-40) with 1 mM PMSF. After 5 times washing the beads with TNE buffer, bounded proteins were separated by 12% SDS-PAGE, lightly stained with Coomassie brilliant blue to verify the fusion proteins, and then visualized by autoradiography.

Immnunohistochemistry, Cytochemistry and Antibody

The GFP-Sizn1 fusion protein was directly detected 42 hours after transfection. Cells were fixed with 3% paraformaldehyde for 5 minutes, counterstained with DAPI (4′,6-Diamidino-2-phenylindole) and detected on a Leica DMR microscope equipped with epifluorescence and a Hammamatsu orca camera. For immunohistochemistry, after transfection, cells were fixed with 3% paraformaldehyde for 15 minutes, permeablized with 0.2% Triton X-100 for 5 minutes, and blocked with PBS containing 1% BSA. Primary antibodies were diluted in PBS and included; anti-myc (Sigma, St. Louis, Mo., 9E10;1/100), anti-PML (Upstate, Waltham, Mass., clone 36.1-104;1/100) and anti-Sizn1 (1/100) antibodies. After washing in PBS, the appropriate secondary antibodies, conjugated to FITC or Texas Red were applied. The cells were washed, counterstained with DAPI and viewed by indirect epifluorescence. Immunolabeled cells were detected with a fluorescent microscope using a fluorescently conjugated secondary antibody (to FITC or Texas Red). The predicted amino acid sequences of Sizn1 were used to design a peptide for generating antibody against Sizn1. The C-terminus (RARARGAPGEPIGLSE; SEQ ID NO: 13) was synthesized and injected to rabbit after conjugation with KLH (keyhole limpet hemocaynin) at Genosys Inc. (The Woodlands, Tex.). This antibody was purified with peptide conjugated beads. Anti-Flag (Sigma, M2;1/500), CBP (Santa Cruz, Santa Cruz, Calif., A-22;1/1000) antibodies were used for immunoblotting analysis.

In situ Hybridization

RNA in situ hybridization on whole-mount embryonic mouse tissue was carried out by using standard methods (Golden et al. (1999) Proc. Natl. Acad. Sci. 96;2439-44; Sossey-Alaoui et al. (1999) Genomics, 60:330-40; Tang et al. (2004) J. Biol. Chem., 279:20369-77). The full-length Sizn1 clone was used as a probe for whole mount in situ hybridization studies. E9.5-E14.5 embryos from CD1 mice were used for all experiments. Riboprobes for Sizn1 were synthesized using Diglabeling kit (Roche).

Northern Blot Analysis

The Adult mouse tissue Northern blotting was performed on blots from Clontech (Mouse MTN blot). Northern Blot analysis was performed using ULTRAhyb® (Ambion, Austin, Tex.) according to the manufacture's instruction and a partial 3′- UTR of Sizn1 as the probe. The glyceraldehyde-3-phosphate dehydrogenase (GAPDH) probe (Clontech) was used as a control. The coding region from the Vesicular acetyl Choline Transporter (VaChT; provided by Dr. Jan Krzysztof Blusztajn; Lopez-Coviella et al. (2000) Science 289:313-6) was used in Northern blots for VaChT.

Immunoprecipitation

After transfection, cells were lyzed in TNE buffer (20 mM Tris-HCl pH 7.4, 150 mM NaCl, 0.5% Triton X-100, 5% glycerol) containing a protease inhibitor cocktail (Roche). For pre-clearance, lysates were incubated with protein-G conjugated beads (Invitrogen). Each primary antibody (2 μg), defined by the experiments was added, incubated at 4° C. for 2 hours, and then incubated with the protein-G conjugated beads for an additional 1 hour. The beads were washed with TNE buffer twice and TNE buffer containing 500 mM NaCl two more times, followed by a final wash in TNE buffer. Bound protein was eluted using SDS-PAGE sample buffer. To detect the associated protein in immunoprecipitate, SDS-PAGE followed by Western blot was performed. Anti-Flag (Sigma, M2;1/500), anti-CBP (Santa Cruz, A-22;1/1000), anti-myc (Sigma, 9E10;1/100) and anti-Sizn1 (1/1000) antibodies were used for immunoblotting analysis.

Metabolic Labelling of Proteins and Immunoprecipitation

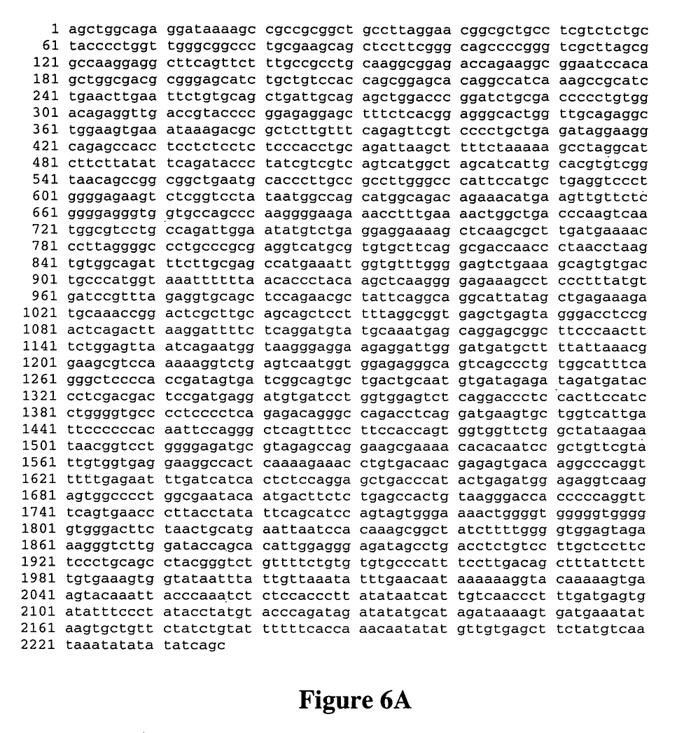

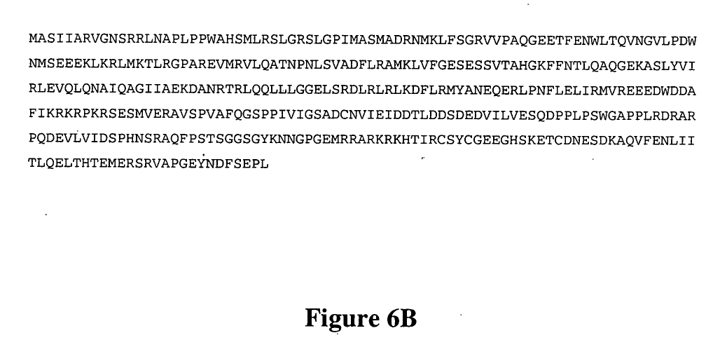

Sizn1 half-life was determined by immunoprecipitation of pulse-labeled proteins. After transfection of expression constructs on 100 mm dish with C2C12, cells were divided to six 60 mm dish one day later. Cells (30% confluent in 60 mm dishes) were incubated with 50 μCi of ³⁵S-labelled methionine/cystine (Amersham, Piscataway, N.J.) in 1 ml of methionine/cystine-free DMEM (Invitrogen) for 2 hours at 37° C. to pulse label Sizn1 proteins. After this time, the medium was replaced with fully supplemented, unlabelled DMEM and an initial sample taken to determine the incorporation of the ³⁵S-labelled methionine/cysteine at the start of the experiment. Five further samples were taken during the next 18 hours chase period as showed in FIG. 6B. Immunoprecipitation experiments were carried out as mentioned above. Gels were dried under a vacuum after fixing in methanol-acetic acid-glycerol and exposure on X-ray film. The band intensities corresponding to Sizn1 or its mutant were calculated with NIH image software. The Sizn1 half-life was calculated by linear regression analysis of log (intensity per band) against time.

Incorporation PCR Single-Strand Conformation Polymorphism (SSCP) (IPS)

Genomic DNA was isolated from lymphocytes using a high salt procedure (Schwartz et al. (1990) Am. J. Hum. Genet., 47:454-8). IPS was performed using 10 ng of genomic DNA in a total volume of 10 μl containing 1×PCR reaction buffer (10 mmol/1 Tris, 50 mmol/l KCl, 1.5 mmol/1 MgC12 pH 8.3) with 1 μmol/1 of each primer, 50 μmol/l of dNTPs, 0.05 μCi of α-³²p dCTP (3000 Ci/mmol, PE), and 0.5 units of Go Taq DNA polymerase (Promega) using a DNA engine thermocycler (M J Research, Waltham, MA) (Sossey-Alaoui et al. (199) Genomics, 60:330-40). The single exon gene was covered with 4 overlapping primer pairs providing complete coverage of the coding sequence (SIZN1-1F/SIZN1-1R: 5′-TACCCTATCGTCGTCAGT-3′ (SEQ ID NO: 14) and 5′-ATGGCTCGCAAGAAATC-3′ (SEQ ID NO: 15); SIZN1-2F/SIZN1-2R: 5′-GGCGACCAACCCTAACCTAAGT-3′ (SEQ ID NO: 16) and 5′-GCCGCTCCTGCTCATTTG-3′ (SEQ ID NO: 17); SIZN1-3F/SIZN1-3R: 5′-TAAGGATTTTCTCAGGATGTATG-3′ (SEQ ID NO: 18) and 5′-GGAATCAATGACCAGCACTT-3′ (SEQ ID NO: 19); SIZN1-4F/SIZN1-4R: 5′-TCAGAGACAGGGCCAGACC-3′ (SEQ ID NO: 20) and 5′-GGCCACTCTTGACCTCTCCAT-3′ (SEQ ID NO: 21)). PCR conditions were as follows: initial denaturation (940 C, 3 minutes), followed by 30 cycles of denaturation (94° C., 20 seconds), annealing (60° or 55° C., 30 s), extension (72° C., 30 seconds), and final extension (72° C, 5 minutes). Following PCR, 10 μl of IPS loading dye (95% formamide, 10 mmol/1 NaOH, 0.25% bromophenol blue, 0.25% xylene cyanol) was added, the samples were denatured at 95° C. for 5 minutes and loaded on a 0.5×MDE gel (FMC) prepared in 0.6×TBE. The gel was run at eight W for 20-24 hours at room temperature, dried and visualized using the BIOMAX™ MS films (Eastman Kodak, Rochester, N.Y.).

Sequence Analysis

Abnormally migrating PCR products on IPS were directly purified using Quantum Prep (Bio-Rad, Hercules, CA) according to the manufacturer's protocol, and bidirectionally sequenced on a MegaBACE (Amersham Biosciences) using the DYEnamic dye terminator kit (Amersham Bioscience) according to the manufacture's protocol. Sequencing primers were the same as those used in the PCR. Sequence alignment and analysis was done in Lasergene® (DNASTAR, Madison, Wis.).

Allele Specific PCR

In order to test if the change was a rare polymorphism, DNA from normal controls were screened using Allele Specific PCR (Papp et al. (2003) Biotechniques, 34:1068-72). Primer SIZN1-MUT (5′-TGGCTAGCATCATTGGAT-3′ (SEQ ID NO: 22)) was utilized in conjunction with primer SIZN1-1R (see primer pairs above) to amplify only the allele containing the alteration. Primer SIZN1-MUT, in addition to having a T as the last nucleotide, the third to last nucleotide was changed from a C to a G. This increased the specificity of the amplification of the mutant allele (Papp et al. (2003) Biotechniques, 34:1068-72). Additionally, control primers were designed at the ACE2 locus on the X chromosome (5′-CACAAAGGTGACAATGG-3′ (SEQ ID NO: 23) and 5′-GTTCTGTGAGACTGTTCATC-3′ (SEQ ID NO: 24)). This allowed for a duplex reaction to be run which contained an internal control. PCR conditions were as given above with an annealing temperature of 55° C. Products were visualized using on a 1% agarose gel.

Primary Septum Culture and Real Time RT-PCR

Dissociated cells (1.5×10⁶ cells per each well) obtained from mouse E14.5 septum were treated with trypsin for 20 minutes, plated on poly-L-lysine/laminin-coated 6 wells plates and incubated in 10% fetal bovine serum-DMEM in presence of FGF (20 ng/ml). To make lentivirus (pLL3.7) containing shRNA against Sizn1, annealed oligonucleotides were subcloned to pLL3.7 vector and pLL3.7 including Sizn1 shRNA sequence (5′-GGAGGAAAAGGTCAGACGT-3′ (SEQ ID NO: 25), A8.2 and Mokola envelope expression constructs were transfected to HEK293T cells (Rubinson et al. (2003) Nat. Genet., 33:401-406). The supernatant was centrifuged and filtered after 2 days. The virus was concentrated by centrifugation at 2,4000 rpm for 80 minutes and resupended in PBS. Primary cells were infected with lentivirus 2 hours after plating. After incubating for 5 days with bFGF (20 ng/ml) ±BMP-2 (10 ng/ml), total RNA was extracted by RNeasy mini (Qiagen) and used for real time PCR and Northern blot analysis to measure Sizn1, ChAT and VaChT mRNA levels. Real time RT-PCR was performed with SYBR green PCR reagent (Qiagen) and ABI PRISM 7000 sequence detection system. Total RNA extracted was used in 4 independent reactions for each real time RT-PCR reaction. Each PCR reaction was done in triplicate. All mRNA levels were normalized to 18S rRNA level. The PCR primers were used as follows: Sizn1 forward (5′-TTTGGTGCAGCTGGTTGTAG-3′; SEQ ID NO: 26) and Sizn1 reverse (5′-ACACGAAGTTCAAGGCGTTT-3′; SEQ ID NO: 27), ChAT forward (5′-AGGGCAGCCTCTCTGTATGA3′; SEQ ID NO: 28) and CHAT reverse (5′-GAGACGGCGGAAATTAATGA3′; SEQ ID NO: 29).

Results and Discussion

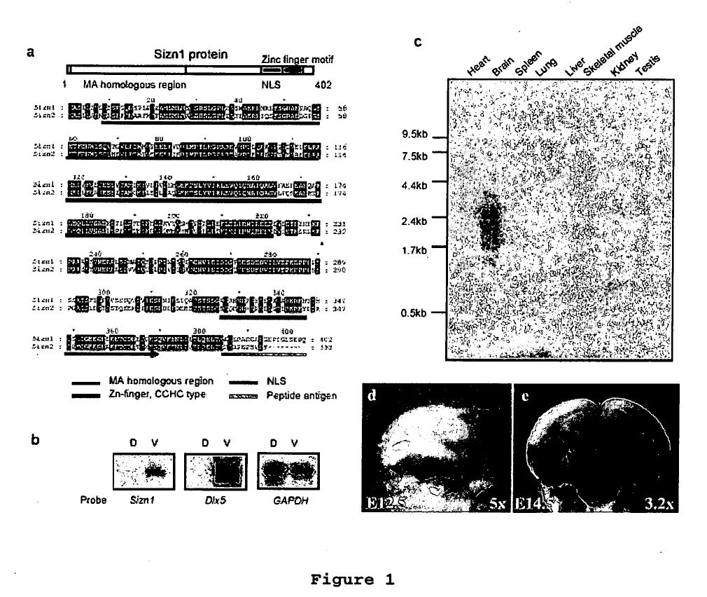

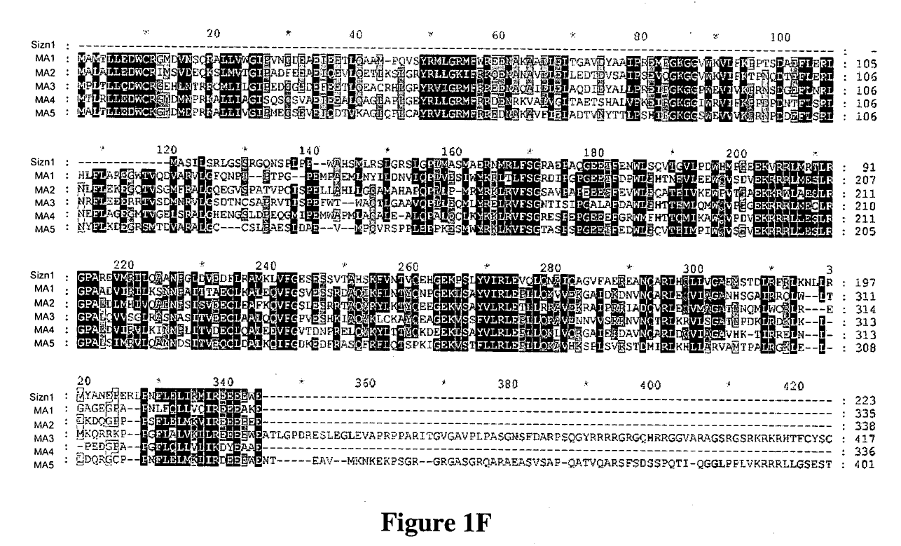

To identify additional genes involved in dorsal-ventral forebrain patterning, a PCR-based subtractive hybridization was performed using pooled samples of dorsal and ventral (basal) mouse prosencephalon microdissected from E11.5 or 12.5 embryos. A dot blot hybridization was employed as an initial screen for differentially expressed transcripts. The differential dorsal or ventral expression of isolated cDNAs was confirmed by Northern blot analysis (FIG. 1B). Sequencing and subsequent cloning from a mouse E12.5 cDNA library of one differentially expressed transcript uncovered a novel gene expressed exclusively in the ventral nervous system (FIG. 1A-B). A tissue Northern blot from adult mice showed strong expression in the brain with low levels of expression in the testis (FIG. 1C). The predicted protein from the 2.4 kb cDNA consists of 402 amino acids and contains an N-terminal MA homologous region, a single classic zinc finger motif (C₂HC type) and a putative nuclear localization sequence (FIG. 1A). Based on these findings, the gene was named Sizn1 (Smad Interacting Zinc finger protein expressed in the Nervous system, see below for Smad interactions: GenBank AY466375).

A database search identified a region of Sizn1 with homologies to the paraneoplastic antigen family (MA1, 2, 3 and 5) and MAP-1 (Modulator of apoptosis:MA4) (Dalmau et al. (1999) Brain Pathol., 9:275-84; Dalmau et al. (1999) Brain, 122(Pt 1):27-39; Dalmau et al. (1999) Arch. Neurol., 56:405-8). The C-terminal regions of these proteins have approximately 50-60% similarity with the N-terminal region of Sizn1 (FIG. 1F). There are at least 5 MA proteins related to Paraneoplastic Syndromes (Dalmau et al. (1999) Brain Pathol., 9:275-84; Dalmau et al. (1999) Arch. Neurol., 56:405-8). Expression of MA proteins appears to be restricted primarily to neurons and spermatogenic cells (Dalmau et al. (1999) Brain, 122(Pt 1):27-39), similar to the expression of Sizn1 (FIG. 1C). Relatively little is known regarding the function of such proteins.

Using the mouse genome database, Sizn1 and a homologous gene (Sizn2: GenBank AY650116) were localized to the X chromosome. Using the Ensemble database, Sizn1 was further mapped to X A3.1, whereas Sizn2 mapped to chromosome X F1. These genes have 76% homology along their entire coding sequence (FIG. 1A). The human ortholog of Sizn1 was identified by homology and found to map to Xq24 (the nucleotide and amino acid sequences of human Sizn1 are provided in FIGS. 6A and 6B, respectively). Mouse Sizn1 has 78% homology to human SIZN1. A human ortholog of Sizn2, based on domain structure; was also identified on human chromosome Xq22.2. However, homologous genes were not found in the worm or fruit fly genomes, indicating the Sizn1 gene is a vertebrate specific gene.

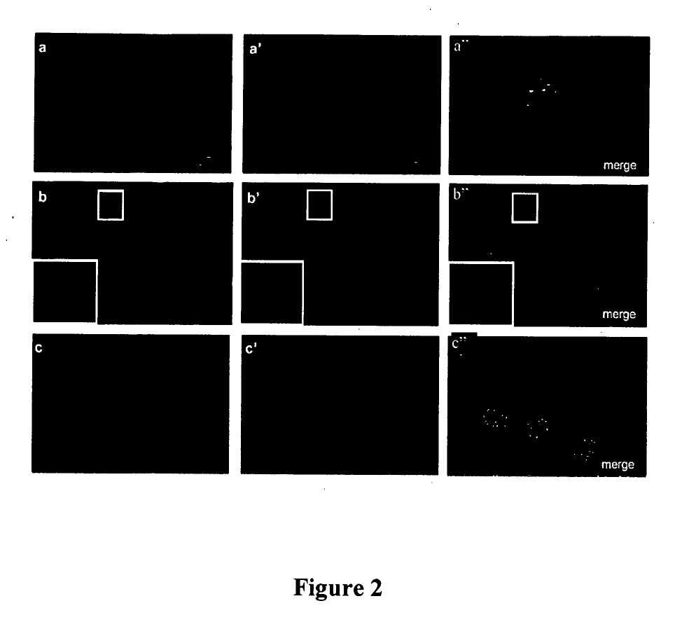

An antibody to Sizn1 was generated against the unique amino acid sequence from the C-terminal region (FIG. 1 and Methods). Immunohistochemistry performed on E12.5 mouse forebrain sections showed Sizn1 to be expressed in an identical pattern to Sizn1 mRNA. (FIG. 1D-E). Immunofluorescence studies on P3 neonate brain (FIG. 2B) as well as localization of a Sizn1-GFP fusion protein with HEK293 cell line (FIG. 2A) demonstrated Sizn1 was localized in a nuclear paraspeckle structure. However, nucleic acid binding studies showed Sizn1 had no direct affinity to DNA or RNA. Co-labeling studies indicate Sizn1 is localized in a large intranuclear protein complex, with presumptive roles in transcription, known as ProMyelocytic Leukemia (PML) bodies (FIG. 2C; Johnson et al. (2004) Annu. Rev. Biochem., 73:355-82; Wang et al. (2004) J. Cell. Biol., 164:515-26; Kiesslich et al. (2002) J. Struct. Biol., 140:167-79). These data suggest Sizn1 may have a role in transcriptional regulation.

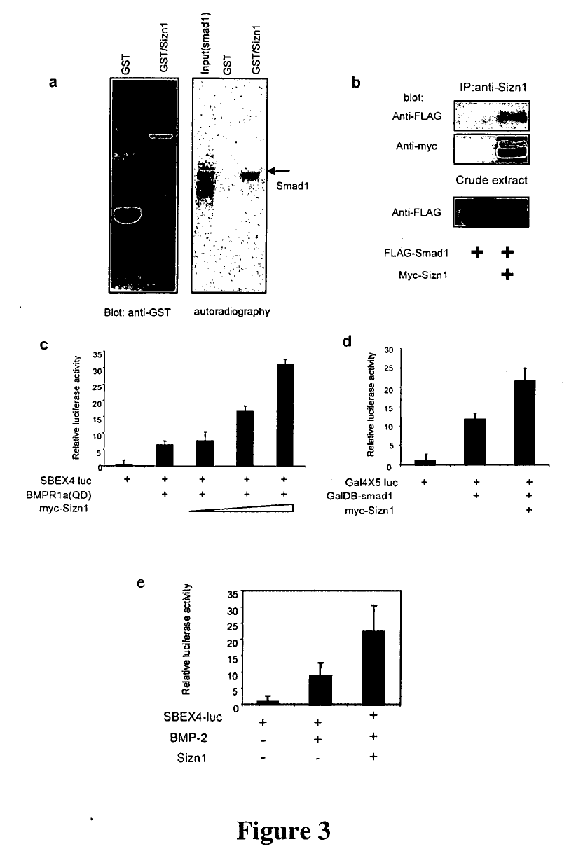

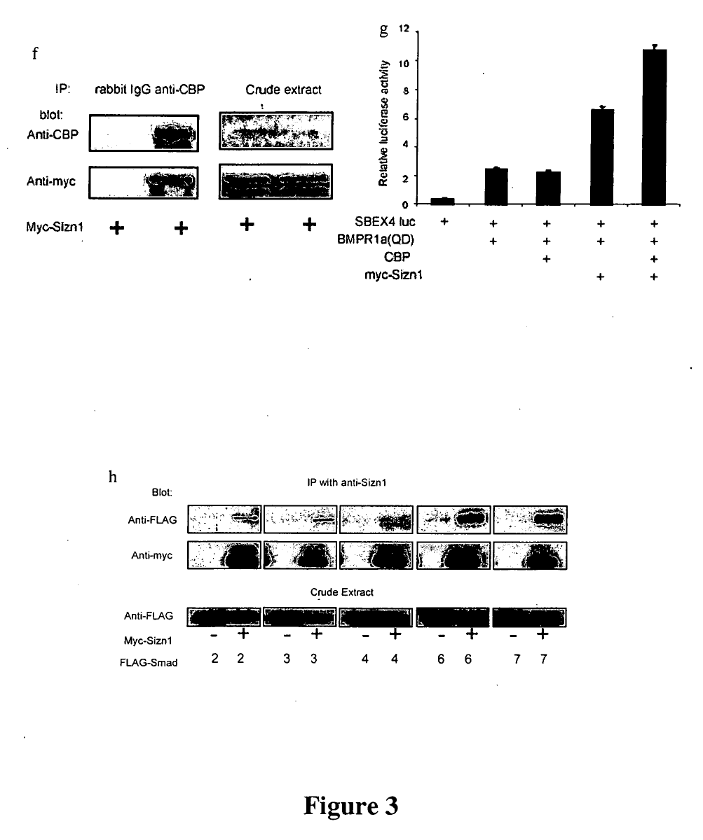

To identify Sizn1 interacting proteins, a yeast two-hybrid screen was performed using Sizn1 as a bait. 5×10⁶ clones from an E11 mouse library (Clontech, GAL4 BD Matchmaker library) were screened. Most of the positive clones were nuclear proteins including members of the Smad family, such as amino acids 382-495 of Smad6 corresponding to the MH2 domain of Smad6. The MH2 domain, in contrast to the DNA binding MH1 domain, has high homologies with other Smad family members (Miyazawa et al. (2002) Genes Cells, 7:1191-204; Shi et al. (2003) Cell, 113:685-700). Both GST-pull down analysis and immunoprecipitation studies confirmed Sizn1 could bind to all Smad family members (FIGS. 3A, 3B, and 3H).

Given the role of Smads in Bone Morphogenic protein (BMP) and TGF-β signaling (Miyazawa et al. (2002) Genes Cells, 7:1191-204; Shi et al. (2003) Cell, 113:685-700) and the clear interactions of Sizn1 with Smad1, the function of Sizn1 in BMP signaling was investigated using a previously characterized in vitro assay (Yoshida et al. (2000) Cell, 103:1085-97). Transfection of the SBEx4-luciferase reporter construct (kindly provided by Dr. T. Yamamoto) into C2C12 cells results in basal level luciferase activity, whereas co-transfection with a constitutively active BMPR1a (QD; referring to the glutamine to aspartic acid substitution resulting in the constitute activity (Lim et al. (2005) Mech. Dev., 122:603-620)) or BMPR1b or treatment with recombinant BMP2 upregulates luciferase expression (FIG. 3C and 3E). Co-transfection of the SBEx4-luciferase reporter vector, BMPRla and Sizn1 show a dose dependent activation of luciferase expression (FIG. 3C). These data indicate Sizn1 positively regulates BMP signaling.

R-Smads, such as Smad1, reside in the cytoplasm prior to pathway activation by BMP (or TGF-β) (Miyazawa et al. (2002) Genes Cells, 7:1191-204; Shi et al. (2003) Cell 113:685-700), whereas Sizn1 is localized in the nucleus. Once phosphorylated, R-Smads interact with Smad4 and translocate to the nucleus (Miyazawa et al. (2002) Genes Cells, 7:1191-204; Shi et al. (2003) Cell 113:685-700). To test the nuclear function of Sizn1, a GAL4-DB-Smad1 fusion protein expression construct was generated and co-transfected with a Gal4×5-luciferase reporter construct and Sizn1 expression construct. This resulted in pathway activation (FIG. 3D). This activity could not be repressed by nuclearly expressed Smad6, although cytoplasmically expressed Smad6 can repress Sizn1 activation of the BMP pathway. These findings support Sizn1 functioning in the nucleus to activate BMP signaling through interaction with Smad1 as transcriptional coactivator. Additional studies can be performed to clarify the potential role of interaction between Smad6 and Sizn1.

Transcriptional coactivators function in a multiprotein complex that docks on transcription factors to recruit chromatin-modifying enzymes (CBP/p300 or SWI/SNF family). BMP transcriptional activation is mediated through Smads, which recruit CBP (CREB-Binding Protein) (Pouponnot et al. (1998) J. Biol. Chem., 273:22865-8). Like Sizn1, CBP also exists in nuclear PML bodies (Boisvert et al. (2001) J. Cell. Biol., 152:1099-1106). In order to test whether Sizn1 functions as a coactivator by facilitating CBP recruitment, a CBP expression construct was co-transfected with a SBEx4-luciferase reporter construct. Although CBP alone does not activate luciferase activity in C2C12 cells, the addition of Sizn1 transcriptionally activates signaling when coupled with CBP (FIG. 3G). Sizn1 also co-precipitates with CBP (FIG. 3F), likely through the interaction of the N-terminal region of Sizn1 containing the MA homology domain and the N-terminal domain of CBP, thereby further supporting a functional interaction. Thus, Sizn1 is able to activate Smad mediated transcription through the recruitment of CBP.

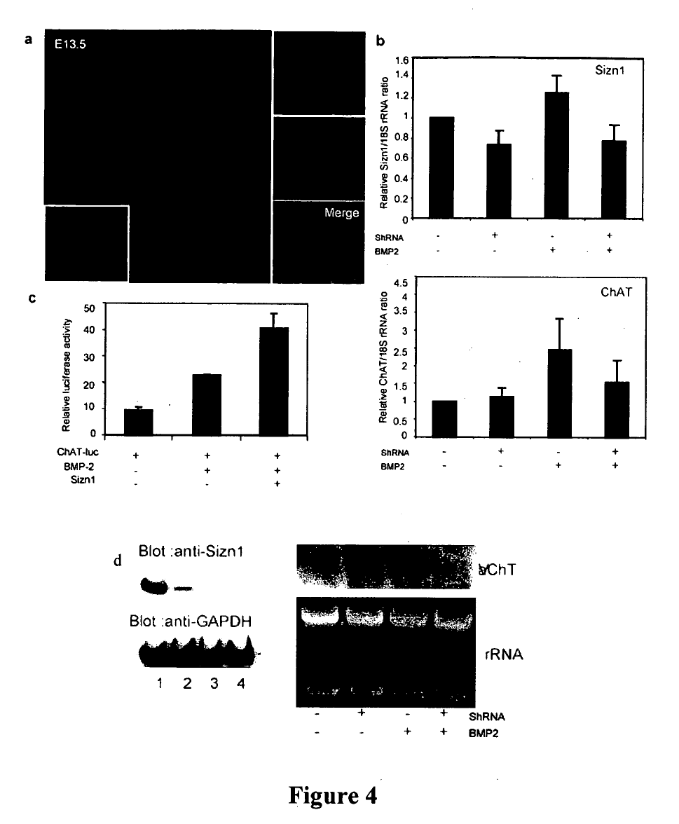

Basal forebrain cholinergic neuron induction and maintenance was recently shown to be dependent on BMP signaling (Lopez-Coviella et al. (2000) Science, 289:313-316; Lopez-Coviella et al. (2005) Proc. Natl. Acad. Sci., 102:6984-9). Sizn1 is expressed in the developing basal forebrain cholinergic neurons (BFCN; FIG. 4). Given that Sizn1 modulates BMP signaling and BMP signalling participates in cholinergic neuron development, Sizn1 may be necessary for cholinergic neuron differentiation. To examine whether Sizn1 is required for cholinergic neuron development, primary septum cells from E13.5 mice were cultured and infected with a lentivirus expressing one of three shRNAs (short hairpin RNAa) against three target sites in Sizn1 (FIG. 4B). The shRNAs to Sizn1 were first tested in SN56 cells, an immortalized mouse septum cell line (Lopez-Coviella et al. (2000) Science, 289:313-316). The target 2 shRNA inhibited expression of Sizn1 efficiently and was used for in the viral infections of primary septum neurons (FIG. 4D). As with the target 2 shRNA (SEQ ID NO: 25; lane 3, FIG. 4D), the target 3 snRNA also inhibited expression of Sizn1 (GGAGACTTTTATTAATCCA; SEQ ID NO: 30; lane 4, FIG. 4D). mRNA levels for Sizn1, ChAT (choline acetyltransferase) and VaChT (vesicular acetylcholine transporter) (FIG. 4B and 4D) were measured by real-time RT-PCR or Northern blot. Sizn1 expression showed a slight induction by BMP and cultures infected with the shRNA to Sizn1 showed a repression of Sizn1 expression (FIG. 4B). ChAT and VaChT was upregulated by BMP and both transcripts were repressed in the presence of shRNA to Sizn1. To support the loss-of-function data, gain-of-function experiments were performed using a ChAT-luciferase reporter construct. The addition of BMP-2 to C2C12 cells transfected with ChAT-luciferase resulted in a modest up-regulation of luciferase activity that was significantly enhanced by the coexpression of Sizn1 (FIG. 4C). These data indicate Sizn1 is necessary for the normal BMP dependent development of basal forebrain cholinergic neurons.

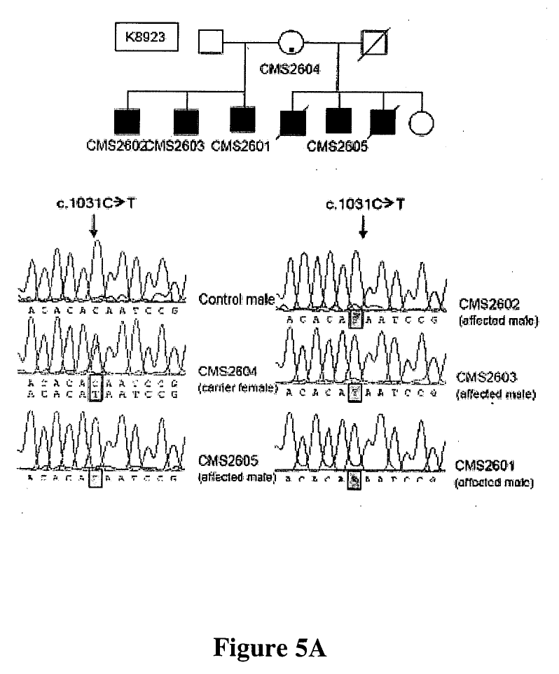

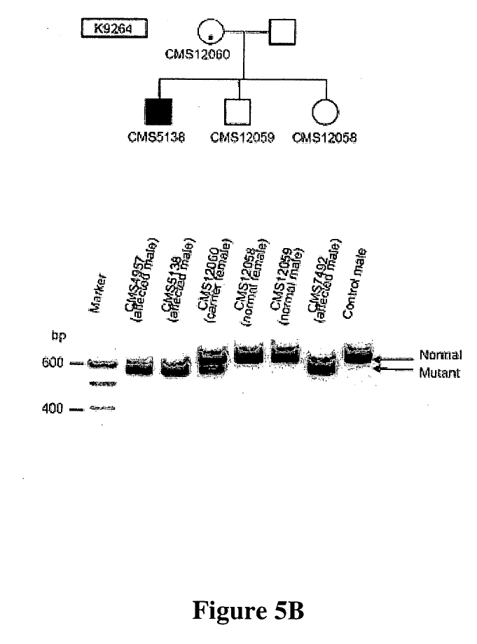

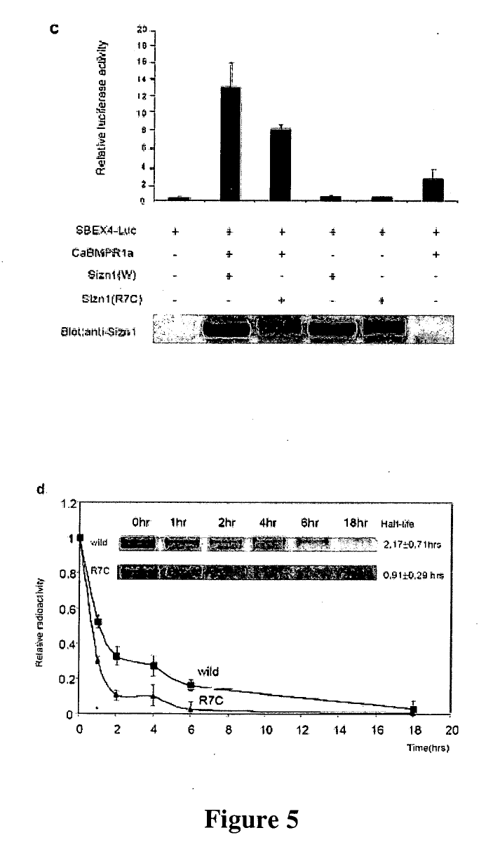

Basal forebrain cholinergic neurons are required for normal cognitive function (Everitt and Robbins (1997) Annu. Rev. Psychol., 48:649-684; Baxter and Chiba (1999) Curr. Opin. Neurobiol., 9:178-183). Based on its location on the X chromosome and expression pattern, Sizn1 might play a role in X-linked mental retardation. 540 males with mental retardation (non-Fragile X) were screened by Incorporation PCR SSCP (Sossey-Alaoui et al. (1999) Genomics, 60:330-40) and suspicious alleles were directly sequenced. Three distinct mutations were identified in 11 of 540 males (2%). Four brothers had a c.1031C>T mutation resulting in p.T433I (FIG. 5A). This allele was not seen in any controls, but the maternal parent for all males was heterozygous for this missense mutation. All four affected males had head circumferences below the 25^{th} percentile (Table 1). A second mutation was found in three affected males, but no controls, which was a 3′UTR deletion.

TABLE 1

| | p.R7C | p.T34411 |
|---|---|---|
| Head circumference ≦25% tile | 0/4 | 4/4 |
| Dysmorphic faces | 3/4 | 0/4 |
| 5^{th} finger clinodactyly | 0/4 | 4/4 |
| Large hands | 0/4 | 4/4 |
| Mild hearing loss | 0/4 | 2/4 |
| Seizures | 3/4 | 0/4 |
| Schizophrenia | 2/4 | 0/4 |
| Behavior problems | 1/4 | 2/4 |
| Moderate mental retardation | 1/4 | 0/4 |
| Mild mental retardation | 3/4 | 4/4 |

A single c.19C>T (p.R7C) mutation was identified in 1.4% (4/290) of unrelated African American males with mental retardation (IQs range from 36 to 63; FIG. 5). The mutant allele was found in 2.9% (5/172) of African American female newborn controls and 3.8% (5/131) of African American female controls with normal intelligence. This mutation had an allele frequency of 1.5% in African American females, and was found to be in Hardy-Weinberg Equilibrium in this group. It has not been found in male controls with normal intelligence (95 African-American male controls and 346 Caucasion males), or male newborns (223 African-Americans and 239 Caucasians). This mutation was also not found in 250 Caucasian males with MR or 405 Caucasian female controls of normal intelligence. Further, immediate relatives of one affected male with the c.19C>T mutation were available for analysis. The mother was determined to be a carrier while the normal male son did not have the c.19C>T allele (FIG. 5B). Based on the observed allele frequency for the c.19C>T change of 1.5% in the African-American population, it should have been found in the African-American male controls (n=318). As this sample size was large enough to detect a 1% polymorphism with 93% power, it is not likely to be a rare polymorphism. Based on the allele frequency data and supported by the observed allele segregation data in family K9264, the c.19C>T allele appears to be a mutant allele and not a polymorphism.

To determine the functional consequence of the c.19C>T (p.R7C) mutation, an expression vector with the c.19C>T (p.R7C) mutation in Sizn1 was generated. Using the SBEx4 luciferase reporter assay, a 40-60% reduction of Sizn1 function was found (FIG. 5C). The R7C mutant protein was also localized onto PML bodies like wild type. However mutant protein levels were found to be significantly reduced in these assays, potentially accounting for the decreased activity (FIG. 5C). Based on these data, the c.19C>T (p.R7C) mutation might effect Sizn1 stability. To examine this possibility, pulse-chase experiments were performed to assess protein half-life. An approximate 50% reduction in Sizn1 half-life was determined. Thus, the functional consequences of this mutation reflect changes in the protein stability and subsequently reduced activity in BMP signaling (FIG. 5D).

This data with the c.19C>T mutation in SIZN1 is curiously similar to that found with several mutations in ATRX. Mutations in this gene result in the ARTX-syndrome characterized by mental retardation, α-thalassemia and developmental abnormalities (Tang et al. (2004) J. Biol. Chem., 279:20369-77; McDowell et al. (1999) Proc. Natl. Acad. Sci., 96:13983-8; Gibbons et al. (2000) Am. J. Med. Genet., 97:204-12). Western blot analyses indicated that the level ATRX expression was significantly reduced in patients with mutations (Tang et al. (2004) J. Biol. Chem., 279:20369-77; McDowell et al. (1999) Proc. Natl. Acad. Sci., 96:13983-8; Gibbons et al. (2000) Am. J. Med. Genet., 97:204-12). Furthermore, ATRX is.localized on PML bodies like Sizn (Tang et al. (2004) J. Biol. Chem., 279:20369-77). Thus, mutations in each of these genes result in mental retardation associated with reduction in protein levels within PML bodies. Additional studies may be performed to better understand the role of the PML body in brain development and function.

In summary, a novel gene, expressed primarily in the ventral neural tube that modulates BMP signaling, has been identified. Sizn1 is localized in PML bodies of the nucleus which function in transcriptional regulation (Johnson et al. (2004) Annu. Rev. Biochem., 73:355-82; Wang et al. (2004) J. Cell. Biol., 164:515-26; Kiesslich et al. (2002) J. Struct. Biol., 140:167-79). Sizn1 is expressed in basal forebrain cholingergic neurons, cells that are dependent on BMP signaling and modulated by Sizn1. Furthermore, defects in basal forebrain cholinergic neurons are associated with cognitive dysfunction (Baxter and Chiba (1999) Curr. Opin. Neurobiol., 9:178-183; Everitt and Robbins (1997) Annu. Rev. Psychol., 48:649-684). Mutational analysis and functional testing indicate that reduced Sizn1 stability and/or function are related in a subset of patients with non-syndromic X-linked mental retardation. Furthermore the data have linked cognitive development with the BMP signaling pathway, an essential pathway in embryonic development.

### EXAMPLE 2

Paraneoplastic neurological diseases (PND) are immune mediated disorders of the nervous system that occur in patients with systemic cancer. Three MA proteins (MA1-3), but not MAP1, are autoantigens of PND (Tan et al. (2001) J. Biol. Chem., 276:2802-7; Rosenfeld et al. (2001) Ann. Neurol., 50:339-48; Dalmau et al. (1999) Brain 122(Pt 1):27-39; Barnett et al. (2001) J. Neurol. Neurosurg. Psychiatry., 70:222-5). Of the 3 MA proteins, the two dominant antigens (Ma1 and Ma2) demonstrate expression patterns restricted primarily to neurons and spermatogenic cells, similar to the expression of Sizn1 (FIG. 1C-1E; Rosenfeld et al. (2001) Ann. Neurol., 50:339-48).

Immunoblot strips containing recombinant Sizn1 were incubated with sera from patients with anti-Ma2 associated encephalitis or the indicated control sera (diluted 1:10,000 in TBST) overnight at 4° C. Strips were then incubated with biotinylated goat anti-human IgG (diluted 1:2,000 in 5% goat serum) for 2 hours at room temperature, avidin-biotin-peroxidase (Vector labs), and the reactivity developed with 0.05% diaminobenzidine tetrahydrochloride (Sigma) with 0.01% hydrogen peroxide. A strip incubated with SIZN1 immunized rabbit serum (diluted 1:1,500) and the appropriate secondary (biotinylated goat anti-rabbit IgG) was used as a positive control. Between steps strips were washed with phosphate buffered saline (PBS).

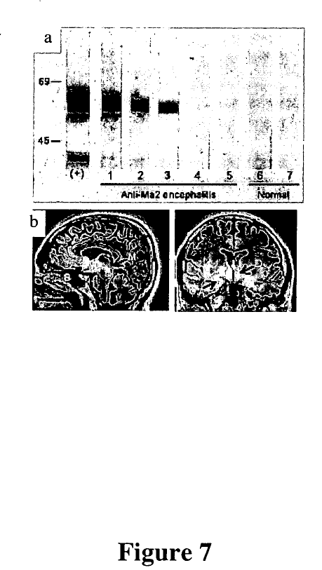

MA2 is recognized by all patients and MA1 by 30% of patients with MA autoimmunity. These data lead to the consideration of Sizn1 as a candidate antigen for PNDs. Serum from 29 patients with MA-related PND, 10 patients with other immune mediated PND, 10 cancer patients without PND, and 10 normal individuals were screened by Western blot using purified GST-Sizn1 protein. The serum from 31% (9/29) patients with MA-related PND reacted with Sizn1 protein with a single band at the expected 55 kD (FIG. 7A). In contrast, none of the other individuals (0/30) had antibodies reacting with Sizn1 protein. Furthermore, the expression of Sizn1 was found to closely parallel distribution of MRI abnormalities in PND patients (FIG. 7B). Indeed, such abnormalities are found in the limbic, diencephalic, and upper mesencephalon, which are the areas of Sizn1 expression. Further, antibodies to Sizn1 were uniquely detected in patients with a PND characteristically associated with severe memory deficits and behavioral and cognitive abnormalities (Dalmau et al. (2004) Brain 127:1831-44). These data implicate Sizn1 as a diagnostic target for PND.

### EXAMPLE 3

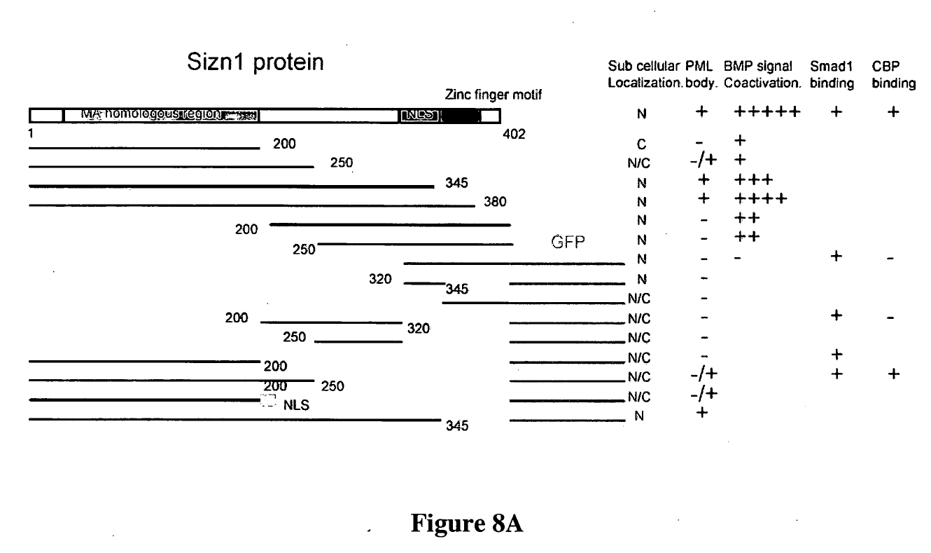

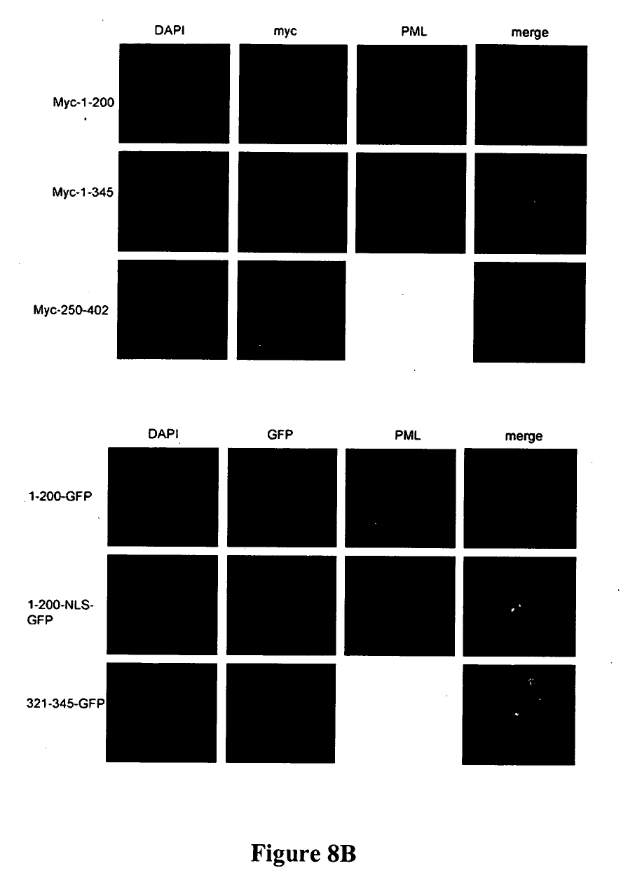

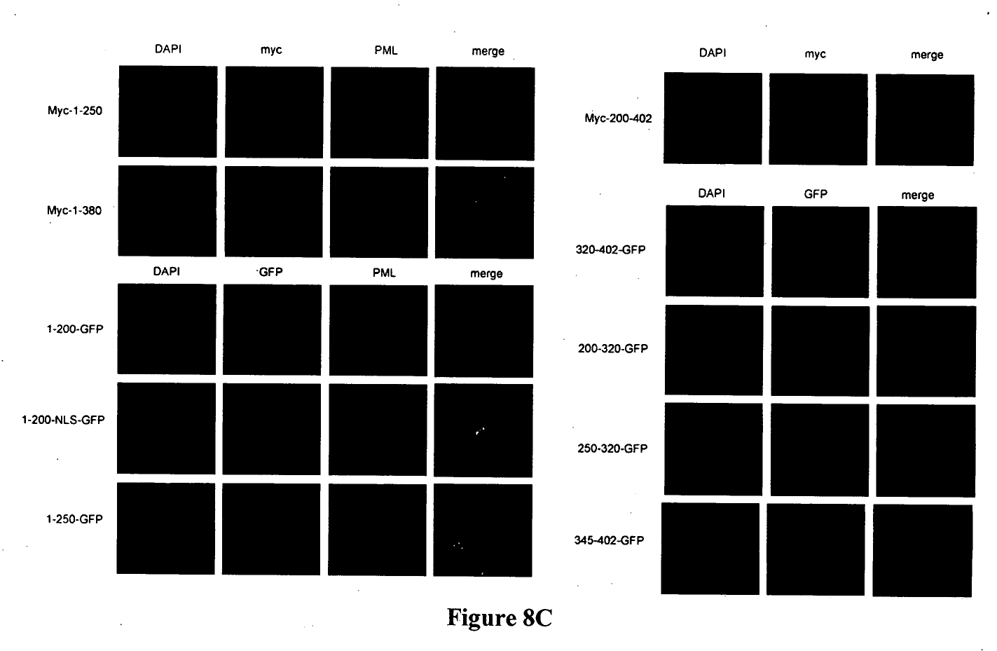

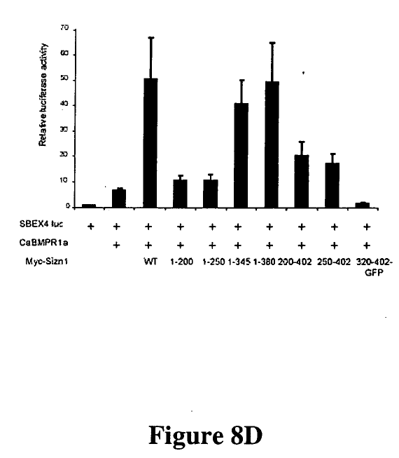

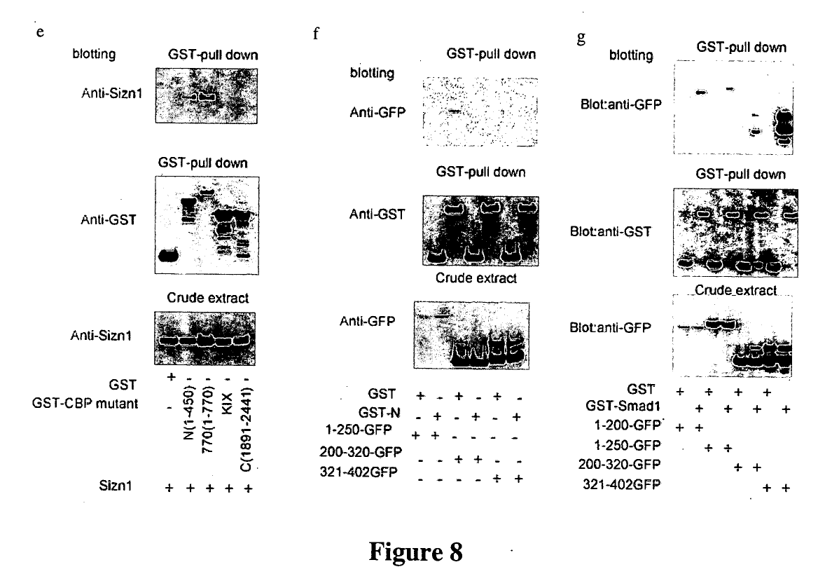

Protein sequence analysis identified a single NLS and a classic Zinc-finger domain (CHCC; FIG. 1A). To begin testing the functions of these domains and other domain in Sizn1, a series of deletion mutants were generated (FIG. 8A). As expected the predicted NLS sequence (aa 320-345), targeted protein to the nucleus, but not to paraspeckles (FIG. 8B and 8C). In contrast, the 1-200 mutant existed only in the cytoplasm while the 1-345 and 1-385 mutants (excluding the zinc finger domain) localized primarily in PML bodies (FIG. 8B and 8C). The 1-250 mutant localized in the cytoplasm and the nucleus (FIG. 8C; within paraspeckles), suggesting a second, but weak, NLS in the 200-250 sequence. Supporting this hypothesis, the 200-320-GFP clone was found to target the protein to the cytoplasm and nucleus (FIG. 8C). These data also suggested the MA homology domain may contain a PML body target sequence. Further support came from N-terminal deletion mutants. Excluding the MA homology domain resulted in nuclear expression, but not in paraspeckles, again supporting the hypothesis that the MA homology domain plays a role in PML body localization. To test this, the 1-200, 1-250 and 1-345 truncated proteins were conjugated with GFP. The 1-250 GFP and 1-345 GFP proteins localized in PML bodies whereas the 1-200 GFP protein did not enter the nucleus as seen for the 1-200 construct (FIGS. 8B and 8C). However, addition of the NLS from SV40 large T antigen to the 1-200 GFP constructs localized the protein to nucleus and PML bodies (FIG. 8B). These data confirm the predicted NLS in Sizn1 and show at least one role for the MA homology domain (1-200) is to localize proteins to nuclear PML bodies.

The functional domain(s) of Sizn1 in BMP signaling were investigated using the deletion constructs described above and the SBEx4-luciferase reporter assay described above (FIG. 8D). The N-terminal mutants, 1-345 and 1-380 retained 80-90% of wild type activity. The C-terminal mutants 200-402 and 250-402 showed far lower pathway activation. These data indicate PML localization of Sizn1 is not sufficient for coactivation of BMP pathway, but required for full coactivation. Interestingly, all deletion mutants showed binding to Smad1 protein in GST-pull down assays (FIG. 8E-8G) indicating binding to Smad1 could not account for reduced levels of BMP signaling (FIG. 8D). The deletion mutants, with the exception of the 320-402 mutant containing zinc finger motif, still bind Smad1 but exhibit reduced signaling capacity, indicating they are unlikely to function as dominant negatives. In conclusion, the weak activation activity of the N-terminal mutants results from loss of CBP binding (see FIGS. 8E-8G), although the possibility that the C-terminus may interact with another transcription co-activator or co-repressor cannot be excluded.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An isolated nucleic acid molecule encoding a Smad Interacting Zinc Finger Protein expressed in the Nervous System (Sizn1), wherein Sizn1 comprises a MA homologous region, a zinc finger motif, and a nuclear localization sequence.

2. The nucleic acid molecule of claim 1, wherein the said Sizn1 is human Sizn1.

3. The nucleic acid molecule of claim 2, wherein the amino acid sequence of said human Sizn1 shares 95% identity with SEQ ID NO: 2.

4. The nucleic acid molecule of claim 3, wherein said amino acid sequence is SEQ ID NO: 2.

5. A nucleic acid probe comprising at least 10 contiguous bases of the nucleic acid molecule encoding a mutant Sizn1 comprising at least one mutation.

6. The nucleic acid probe of claim 5 wherein said mutant Sizn1 encoding nucleic acid molecule has a mutation which replaces a codon encoding arginine with a codon encoding cytosine at position 7 of the amino acid sequence.

7. The nucleic acid probe of claim 6 wherein said mutant Sizn1 encoding nucleic acid molecule has the cytosine at position 19 replaced with a thymine.

8. An isolated protein comprising an amino acid sequence encoded by the nucleic acid molecule of claim 1.

9. An antibody immunologically specific for the protein of claim 8.

10. A method of detecting mental retardation in a patient comprising the steps of:
a) providing a biological sample from said patient, wherein said biological sample comprises nucleic acid molecules; and
b) assessing said nucleic acid molecules for the presence or absence of a mutation in a Sizn1 encoding nucleic acid molecule, wherein said Sizn1 encoding nuclei acid molecule is the nucleic acid molecule of claim 1,
wherein the identification of a mutation in a Sizn1 encoding nucleic acid molecule is correlated with an increased risk that said patient has a mental retardation.

11. The method of claim 10, wherein said mutation results in an arginine to cytosine mutation at position 7 of the encoded amino acid sequence.

12. The method of claim 11, wherein said mutation is a cytosine to thymine at position 19 of the nucleic acid sequence encoding the Sizn1 protein.

13. The method of claim 10, wherein said patient is a fetus.

14. The method of claim 10, wherein said nucleic acid is DNA.

15. The method of claim 10, wherein said nucleic acid is RNA.

16. The method of claim 15, wherein said RNA is mRNA and wherein said mRNA is reverse transcribed into cDNA prior to step b).

17. The method of claim 10, wherein the detection of a mutation in step b) is performed by a method selected from the group consisting of direct sequencing of nucleic acids, conformation sensitive gel electrophoresis, single strand polymorphism assay, denaturation gradient gel electrophoresis, restriction fragment length polymorphism assay, ligase chain reaction, enzymatic cleavage and southern hybridization.

18. A method of detecting mental retardation in a patient comprising the steps of:
a) providing a biological sample form a patient; and
b) measuring the level of Sizn1 protein or fragment thereof in said biological sample, wherein said Sizn1 protein is the protein of claim 8,
wherein a decrease in Sizn1 protein in said biological sample from said patient compared with the amount of Sizn1 protein in a corresponding biological sample obtained from a normal individuals is correlated with an increased risk that said patient has a mental retardation.

19. The method of claim 18, wherein said patient is a fetus.

20. The method of claim 18, wherein said level of Sizn1 protein is measured by an immunodetection assay.

21. A method of detecting mental retardation in a patient comprising the steps of:
a) contacting a biological sample comprising nucleic acid molecules obtained from said patient with the Sizn1 probe of claim 5 under conditions suitable for hybridization;
b) detecting the presence of a hybridization product,
wherein the presence of said hybridization product indicates the presence of the at least one mutation present in the Sizn1 probe in said Sizn1 nucleic acid molecule and thereby the likelihood of mental retardation in the patient.

22. The method of claim 21, wherein said nucleic acid molecules contained in a biological sample of step a) are purified prior to hybridization.

23. The method of claim 21, wherein said nucleic acid molecules contained in a biological sample of step a) are selected from the group consisting of Sizn1 genomic DNA, Sizn1 RNA, and Sizn1 cDNA made from Sizn1 mRNA.

24. A kit for detecting the presence of a mutant Sizn1 encoding nucleic acid in a biological sample, comprising:
a) at least one oligonucleotide which specifically hybridizes with mutant Sizn1 encoding nucleic acid molecules;
b) reaction buffer; and
c) instructional material.

25. The kit as claimed in claim 24, wherein said at least one oligonucleotide contains a tag.

26. A kit for detecting the presence a mutant Sizn1 encoding nucleic acid molecule in a biological sample, comprising:
a) antibodies of claim 9; and
b) instructional material.

27. The kit as claimed in claim 26, wherein said antibody contains a tag.

28. A method for diagnosing paraneoplastic neurological disease in a patient comprising:
a) obtaining a biological sample from said patient; and
b) assaying for the presence of the Sizn1 antibody of claim 9,
wherein the presence of said Sizn1 antibodies in said biological sample is indicative of said paraneoplastic neurological disease.

29. A method for identifying a therapeutic compound for the treatment of a paraneoplastic neurological disease comprising:
a) providing the Sizn1 antibodies of claim 9;
b) providing at least one compound;
c) contacting said Sizn1 antibodies and said at least one compound with a Sizn1 protein or fragment thereof; and
d) determining the level of binding of said Sizn1 antibody with said Sizn1 protein in the presence of said at least one compound, wherein a decrease in the binding of said Sizn1 antibody with said Sizn1 protein or fragment thereof in the presence of said compound indicates said compound is a therapeutic compound.

30. The method of claim 29, wherein said Sizn1 antibody is obtained from a patient having said paraneoplastic neurological disease.


SEQUENCE LISTING (30 sequences)

SEQ ID NO: 1 - DNA, 2237 bp, Artificial Sequence
  Synthetic Sequence
agctggcagaggataaaagccgccgcggctgccttaggaacggcgctgcctcgtctctgctacccctggttgggcggccctgcgaagcagctccttcgggcagccccgggtcgcttagcggccaaggaggcttcagttctttgccgcctgcaaggcggagaccagaaggcggaatccacagctggcgacgcgggagcatctgctgtccaccagcggagcacaggccatcaaagccgcatctgaacttgaattctgtgcagctgattgcagagctggacccggatctgcgaccccctgtggacagaggttgaccgtaccccggagaggagctttctcacggagggcactggttgcagaggctggaagtgaaataaagacgcgctcttgtttcagagttcgtcccctgctgagataggaaggcagagccacctcctctcctctcccacctgcagattaagcttttctaaaaagcctaggcatcttcttatattcagataccctatcgtcgtcagtcatggctagcatcattgcacgtgtcggtaacagccggcggctgaatgcacccttgccgccttgggcccattccatgctgaggtccctggggagaagtctcggtcctataatggccagcatggcagacagaaacatgaagttgttctcggggagggtggtgccagcccaaggggaagaaacctttgaaaactggctgacccaagtcaatggcgtcctgccagattggaatatgtctgaggaggaaaagctcaagcgcttgatgaaaacccttaggggccctgcccgcgaggtcatgcgtgtgcttcaggcgaccaaccctaacctaagtgtggcagatttcttgcgagccatgaaattggtgtttggggagtctgaaagcagtgtgactgcccatggtaaattttttaacaccctacaagctcaaggggagaaagcctccctttatgtgatccgtttagaggtgcagctccagaacgctattcaggcaggcattatagctgagaaagatgcaaaccggactcgcttgcagcagctccttttaggcggtgagctgagtagggacctccgactcagacttaaggattttctcaggatgtatgcaaatgagcaggagcggcttcccaactttctggagttaatcagaatggtaagggaggaagaggattgggatgatgcttttattaaacggaagcgtccaaaaaggtctgagtcaatggtggagagggcagtcagccctgtggcatttcagggctccccaccgatagtgatcggcagtgctgactgcaatgtgatagagatagatgataccctcgacgactccgatgaggatgtgatcctggtggagtctcaggaccctccacttccatcctggggtgcccctcccctcagagacagggccagacctcaggatgaagtgctggtcattgattccccccacaattccagggctcagtttccttccaccagtggtggttctggctataagaataacggtcctggggagatgcgtagagccaggaagcgaaaacacacaatccgctgttcgtattgtggtgaggaaggccactcaaaagaaacctgtgacaacgagagtgacaaggcccaggtttttgagaatttgatcatcactctccaggagctgacccatactgagatggagaggtcaagagtggcccctggcgaatacaatgacttctctgagccactgtaagggaccacccccaggtttcagtgaacccttacctatattcagcatccagtagtgggaaaactggggtgggggtgggggtgggacttctaactgcatgaattaatccacaaagcggctatcttttggggtggagtagaaagggtcttggataccagcacattggagggagatagcctgacctctgtccttgctccttctccctgcagcctacgggtctgttttctgtgtgtgcccatttccttgacagctttattctttgtgaaagtggtataatttattgttaaatatttgaacaataaaaaaggtacaaaaagtgaagtacaaattacccaaatctctccacccttatataatcattgtcaaccctttgatgagtgatatttccctatacctatgtacccagatagatatatgcatagataaaagtgatgaaatataagtgctgttctatctgtattttttcaccaaacaatatatgttgtgagcttctatgtcaataaatatatatatcagc

SEQ ID NO: 2 - protein, 402 aa, Artificial Sequence
  Synthetic Sequence
MASIIARVGNSRRLNAPLPPWAHSMLRSLGRSLGPIMASMADRNMKLFSGRVVPAQGEETFENWLTQVNGVLPDWNMSEEEKLKRLMKTLRGPAREVMRVLQATNPNLSVADFLRAMKLVFGESESSVTAHGKFFNTLQAQGEKASLYVIRLEVQLQNAIQAGIIAEKDANRTRLQQLLLGGELSRDLRLRLKDFLRMYANEQERLPNFLELIRMVREEEDWDDAFIKRKRPKRSESMVERAVSPVAFQGSPPIVIGSADCNVIEIDDTLDDSDEDVILVESQDPPLPSWGAPPLRDRARPQDEVLVIDSPHNSRAQFPSTSGGSGYKNNGPGEMRRARKRKHTIRCSYCGEEGHSKETCDNESDKAQVFENLIITLQELTHTEMERSRVAPGEYNDFSEPL

SEQ ID NO: 3 - protein, 402 aa, Artificial Sequence
  Synthetic Sequence
MASILSRLGSSRGQNSPLPPWAHSMLRSLGRSLGPLMASMAERNMRLFSGRAEPAQGEETFENWLSQVTGVLPDWHMPEEEKVRRLMRTLRGPAREVMRLLQAANPGLDVEDFLRAMKLVFGESESSVTAHSKFVNTVQEHGEKPSLYVIRLEVQLQNAIQAGVFAEREANQARLHQLLVGAEMSTDLRFRLKNLLRVYANEPERLPNFLELIRMIREEEEWEETFINPKRPRRAESVMERALSPMAFQSSPPIMISSIDCNVIEIDDSPDDSDEDVILVEPEDPPLPSSSASSFLGRAVSEDQVLVIESPNIFEIQAPSTSSGAGRKNNRGFGELRRARKRKHTIHCSHCGEEGHSKETCDNESDKGQVFENLIITLQELTHAEERARGAPGEPIGLSEPQ

SEQ ID NO: 4 - protein, 393 aa, Artificial Sequence
  Synthetic Sequence
MASLLARMGNSRRQNAAFMPFAHSMLRALGRSLGPLIANIAERNIQSFSGRAELGPGEETFENWLSQVHEVLPDWPMSEEEKIKRLMRTLRGPAREAMRLFQADNPNLNVAEFLRAMKLLFGASESSITAHGKFLSTLQAQGEKPSLYVIRLEVQLQNAIQAGVLPQSEANRTRLHQLLVGAELSRELRTKLKGLLQMHAHNEQENLPDFLELIRMIREEEDWDETFLRNKRPRRSETVMERAASPVVFQGSLPIVIGSADCNVIEIDDSQDDSDEDVILVEPEDPPLSSPGASSLRGTASTQEEMLIIESPDDFDEESPSTSSGSGQRNNGPGDLGRTRKRKYPIRCPHCGEEGHAKETCDNTSNKGQVFENLIVTLQELTHMERPKPSVPY

SEQ ID NO: 5 - protein, 223 aa, Artificial Sequence
  Synthetic Sequence
MASILSRLGSSRGQNSPLPPWAHSMLRSLGRSLGPLMASMAERNMRLFSGRAEPAQGEETFENWLSQVTGVLPDWHMPEEEKVRRLMRTLRGPAREVMRLLQAANPGLDVEDFLRAMKLVFGESESSVTAHSKFVNTVQEHGEKPSLYVIRLEVQLQNAIQAGVFAEREANQARLHQLLVGAEMSTDLRFRLKNLLRVYANEPERLPNFLELIRMIREEEEWE

SEQ ID NO: 6 - protein, 335 aa, Artificial Sequence
  Synthetic Sequence
MAMTLLEDWCRGMDVNSQRALLVWGIPVNCDEAEIEETLQAAMPQVSYRMLGRMFWREENAKAALLELTGAVDYAAIPREMPGKGGVWKVLPKPPTSDAEFLERLHLFLAREGWTVQDVARVLGFQNPTPTPGPEMPAEMLNYILDNVIQPLVESIWYKRLTLFSGRDIPGPGEETFDPWLEHTNEVLEEWQVSDVEKRRRLMESLRGPAADVIRILKSNNPAITTAECLKALEQVFGSVESSRDAQIKFLNTYQNPGEKLSAYVIRLEPLLQKVVEKGAIDKDNVNQARLEQVIAGANHSGAIRRQLWLTGAGEGPAPNLFQLLVQIREEEAKE

SEQ ID NO: 7 - protein, 338 aa, Artificial Sequence
  Synthetic Sequence
MALALLEDWCRIMSVDEQKSLMVTGIPADFEEAEIQEVLQETLKSLGRYRLLGKIFRKQENANAVLLELLEDTDVSAIPSEVQGKGGVWKVIPKTPNQTEFLERLNLFLEKEGQDTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEE

SEQ ID NO: 8 - protein, 417 aa, Artificial Sequence
  Synthetic Sequence
MPLTLLQDWCRGEHLNTRPCMLILGIPEDCGEDEFEETLQEACRHLGRYRVIGRMFRREENAQAILLELAQDIDYALLPREIPGKGGPWEVIVKPRNSDGEFLNRLNRFLEEERRTVSDMNRVLGSDTNCSAPRVTISPEFWTWAQTLGAAVQPLLEQMLYRELRVFSGNTISIPGALAFDAWLEHTTEMLQMWQVPEGEKRRRLMECLRGPALQVVSGLRASNASITVEECLAALQQVFGPVESHKIAQVKLCKAYQEAGEKVSSFVLRLEPLLQRAVENNVVSRRNVNQTRLKRVLSGATLPDKLRDKLKLMKQRRKPPGFLALVKLLREEEEWEATLGPDRESLEGLEVAPRPPARITGVGAVPLPASGNSFDARPSQGYRRRRGRGCHRRGGVARAGSRGSRKRKRHTFCYSC

SEQ ID NO: 9 - protein, 336 aa, Artificial Sequence
  Synthetic Sequence
MTLRLLEDWCRGMDMNPRKALLIAGISQSCSVAEIEEALQAGLAPLGEYRLLGRMFRRDENRKVALVGLTAETSHALVPKEIPGKGGIWRVIFKPPDPDNTFLSRLNEFLAGEGMTVGELSRALGHENGSLDPEQGMIPEMWAPMLAQALEALQPALQCLKYKKLRVFSGRESPEPGEEEFGRWMFHTTQMIKAWQVPDVEKRRRLLESLRGPALDVIRVIKINNPLITVDECLQALEEVFGVTDNPRELQVKYLTTYQKDEEKLSAYVLRLEFLLQKLVQRGAIERDAVNQARIDQVIAGAVHKTIRRELNLPEDGPAPGFLQLLVLIKDYEAAE

SEQ ID NO: 10 - protein, 401 aa, Artificial Sequence
  Synthetic Sequence
MALTLLEDWCKGMDMDPRKALLIVGIPMECSEVEIQDTVKAGLQPLCAYRVLGRMFRREDNAKAVEIELADTVNYTTLPSHIPGKGGSWEVVVKPRNPDDEFLSRLNYFLKDEGRSMTDVARALGCCSLPAESLDAEVMPQVRSPPLEPPKESMWYRKLKVFSGTASPSPGEETFEDWLEQVTEIMPIWQVSEVEKRRRLLESLRGPALSIMRVLQANNDSITVEQCLDALKQIFGDKEDFRASQFRFIQTSPKIGEKVSTFLLRLEPLLQKAVHKSPLSVRSTDMIRLKHLLARVAMTPALRGKLELLDQRGCPPNFLELMKLIRDEEEWENTEAVMKNKEKPSGRGRGASGRQARAEASVSAPQATVQARSFSDSSPQTIQGGLPPLVKRRRLLGSEST

SEQ ID NO: 11 - DNA, 12 bp, Artificial Sequence
  Synthetic Sequence
acacacaatccg

SEQ ID NO: 12 - DNA, 12 bp, Artificial Sequence
  Synthetic Sequence
acacataatccg

SEQ ID NO: 13 - protein, 16 aa, Artificial Sequence
  Synthetic Sequence
RARARGAPGEPIGLSE

SEQ ID NO: 14 - DNA, 18 bp, Artificial Sequence
  Synthetic Sequence
taccctatcgtcgtcagt

SEQ ID NO: 15 - DNA, 17 bp, Artificial Sequence
  Synthetic Sequence
atggctcgcaagaaatc

SEQ ID NO: 16 - DNA, 22 bp, Artificial Sequence
  Synthetic Sequence
ggcgaccaaccctaacctaagt

SEQ ID NO: 17 - DNA, 18 bp, Artificial Sequence
  Synthetic Sequence
gccgctcctgctcatttg

SEQ ID NO: 18 - DNA, 23 bp, Artificial Sequence
  Synthetic Sequence
taaggattttctcaggatgtatg

SEQ ID NO: 19 - DNA, 20 bp, Artificial Sequence
  Synthetic Sequence
ggaatcaatgaccagcactt

SEQ ID NO: 20 - DNA, 19 bp, Artificial Sequence
  Synthetic Sequence
tcagagacagggccagacc

SEQ ID NO: 21 - DNA, 21 bp, Artificial Sequence
  Synthetic Sequence
ggccactcttgacctctccat

SEQ ID NO: 22 - DNA, 18 bp, Artificial Sequence
  Synthetic Sequence
tggctagcatcattggat

SEQ ID NO: 23 - DNA, 17 bp, Artificial Sequence
  Synthetic Sequence
cacaaaggtgacaatgg

SEQ ID NO: 24 - DNA, 20 bp, Artificial Sequence
  Synthetic Sequence
gttctgtgagactgttcatc

SEQ ID NO: 25 - DNA, 19 bp, Artificial Sequence
  Synthetic Sequence
ggaggaaaaggtcagacgt

SEQ ID NO: 26 - DNA, 20 bp, Artificial Sequence
  Synthetic Sequence
tttggtgcagctggttgtag

SEQ ID NO: 27 - DNA, 20 bp, Artificial Sequence
  Synthetic Sequence
acacgaagttcaaggcgttt

SEQ ID NO: 28 - DNA, 20 bp, Artificial Sequence
  Synthetic Sequence
agggcagcctctctgtatga

SEQ ID NO: 29 - DNA, 20 bp, Artificial Sequence
  Synthetic Sequence
gagacggcggaaattaatga

SEQ ID NO: 30 - DNA, 19 bp, Artificial Sequence
  Synthetic Sequence
ggagacttttattaatcca